(19) United States
(12) Patent Application Publication
(10) Pub. No.: US 2025/0147048 A1
(43) Pub. Date: May 8, 2025

(54) METHODS OF DIAGNOSTIC SCREENING AND EARLY DETECTION OF ADENOMYOSIS

(71) Applicant: ARIZONA BOARD OF REGENTS ON BEHALF OF THE UNIVERSITY OF ARIZONA, Tucson, AZ (US)

(72) Inventors: Melissa M. Herbst-Kralovetz, Phoenix, AZ (US); Pawel Laniewski, Phoenix, AZ (US)

(21) Appl. No.: 19/018,644

(22) Filed: Jan. 13, 2025

Related U.S. Application Data
(63) Continuation-in-part of application No. PCT/US23/70167, filed on Jul. 13, 2023.
(60) Provisional application No. 63/388,823, filed on Jul. 13, 2022.

(51) Int. Cl. G01N 33/68

(52) U.S. Cl. CPC G01N 33/6893; G01N 33/6806; G01N 33/6863; G01N 2570/00; G01N 2800/364

(57) ABSTRACT

Adenomyosis is a highly prevalent yet enigmatic disease that can be diagnosed only via histopathology among women undergoing hysterectomy, thus, leading to underdiagnosis. Unfortunately, research on adenomyosis has been limited due to a variety of challenges, and little work has been done on non-invasive diagnostics for adenomyosis. Currently, there is no FDA-approved non-invasive method for early detection of adenomyosis. By integrating immunoassays and metabolomics analyses, candidate biomarkers for the non-invasive diagnosis of adenomyosis in cervicovaginal lavages have been identified. Thus, methods for non-invasive diagnostic, screening, and early detection of adenomyosis in patients are described herein.

### CROSS-REFERENCES TO RELATED APPLICATIONS

This application is a continuation-in-part and claims benefit of PCT Application No. PCT/US23/70167 filed Jul. 13, 2023, which claims benefit of U.S. Provisional Application No. 63/388,823 filed Jul. 13, 2022, the specifications of which are incorporated herein in their entirety by reference.

### FIELD OF THE INVENTION

The present invention features methods for diagnostic screening and early detection of adenomyosis in patients.

### BACKGROUND OF THE INVENTION

Adenomyosis is a highly prevalent yet enigmatic disease that can be diagnosed only via histopathology among women undergoing hysterectomy, thus, leading to underdiagnosis. Research progress on adenomyosis has been limited due to a variety of challenges: 1) adenomyosis lacks both a clear etiology and clinical/pathological phenotyping, 2) there are no established diagnostic guidelines, 3) there are no clear criteria for assessment of treatment response, and 4) adenomyosis co-occurs with other gynecologic conditions. Based on the challenges outlined, little work has been done on non-invasive diagnostics for adenomyosis, and currently, there is no FDA-approved non-invasive method for early detection of adenomyosis, as it is only diagnosed by surgical intervention. Thus, there is an urgent and unmet need for identifying novel diagnostic biomarkers and pathophysiological mechanisms based on non-invasive sampling, which could lead to earlier intervention and treatment.

The present invention features non-invasive sampling that allows for the detection of adenomyosis-related metabolites and proteins in the cervicovaginal microenvironment. The present invention has identified candidate biomarkers for the non-invasive diagnosis of adenomyosis in cervicovaginal lavages by integrating immunoassays and metabolomics analyses.

### BRIEF SUMMARY OF THE INVENTION

It is an objective of the present invention to provide methods that allow for non-invasive diagnosis of adenomyosis in cervicovaginal lavages by integrating immunoassays and metabolomics analyses, as specified in the independent claims. Embodiments of the invention are given in the dependent claims. Embodiments of the present invention can be freely combined with each other if they are not mutually exclusive.

In some embodiments, the present invention features a non-invasive method of diagnosing a benign gynecologic condition (e.g., adenomyosis) in a patient. The method may comprise determining the patient's levels of five or more biomarkers comprising protein biomarkers, metabolite biomarkers, or a combination thereof. The level of the five or more biomarkers can be determined by obtaining a biological sample (e.g., a cervicovaginal lavage (CVL) sample or a vaginal swab sample) from the patient and measuring the levels of five or more biomarkers in the sample obtained. In some embodiments, if the levels of at least five or more biomarkers are altered from a predetermined threshold, then the patient is diagnosed with a benign gynecologic condition (e.g., adenomyosis). In other embodiments, the patient is diagnosed with a benign gynecologic condition (e.g., adenomyosis) if the levels of at least five or more biomarkers are altered from a control patient (i.e., patients without a benign gynecologic condition). In certain embodiments, the the patient is diagnosed with a benign gynecologic condition (e.g., adenomyosis) if the levels of at least five or more biomarkers are upregulated (e.g., from a predetermined threshold or a control patient). In some embodiments, the five or more biomarkers are selected from a group consisting of, consisting essentially of, or comprising comprise N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, phenethylamine, or a combination thereof.

In some embodiments, the present invention features a method comprising obtaining a biological sample (e.g., a CVL sample or a vaginal swab sample) from a patient, producing a profile of the biological sample (e.g., a CVL sample) collected by detecting at least five or more biomarkers comprising protein biomarkers, metabolite biomarkers or a combination thereof and analyzing the biological sample profile produced.

In some embodiments, the present invention may also feature a method of treating adenomyosis in a patient in need thereof. The method may comprise diagnosing the patient with adenomyosis with methods described herein and administering a therapeutic amount of a treatment to the patient if the patient is diagnosed with adenomyosis.

In other embodiments, the present invention features a method of distinguishing between adenomyosis and endometriosis in a subject. The method may comprise obtaining a biological sample (e.g., a CVL sample or a vaginal swab sample) from the subject and measuring the levels of five or more biomarkers in the sample obtained. In some embodiments, the patient is diagnosed with adenomyosis if the levels of at least five or more biomarkers are altered from a predetermined threshold. In other embodiments, the patient is diagnosed with adenomyosis if the levels of at least five or more biomarkers are altered from a control patient (i.e., patients without adenomyosis). In certain embodiments, the the patient is diagnosed with a benign gynecologic condition (e.g., adenomyosis) if the levels of at least five or more biomarkers are upregulated (e.g., from a predetermined threshold or a control patient). In some embodiments, the five or more biomarkers are selected from a group consisting of, consisting essentially of, or comprising comprise N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, phenethylamine, or a combination thereof.

In other embodiments, the present invention may feature an in vitro method of diagnosing adenomyosis in a subject in need thereof. The method may comprise producing a profile from a biological sample (e.g., a CVL sample or a vaginal swab sample) obtained from the subject by detecting at least five or more biomarkers and analyzing the biological sample profile produced. In some embodiments, the patient is diagnosed with adenomyosis if the levels of at least five or more biomarkers are altered from a predetermined threshold. In other embodiments, the patient is diagnosed with adenomyosis if the levels of at least five or more biomarkers are altered from a control patient (i.e., patients without adenomyosis).

One of the unique and inventive technical features of the present invention is using samples collected using cervicovaginal lavage (CVL). Without wishing to limit the invention to any theory or mechanism, it is believed that the technical feature of the present invention advantageously provides a non-invasive method for diagnosing patients with adenomyosis. None of the presently known prior references or work has the unique, inventive technical feature of the present invention.

Furthermore, the prior references teach away from the present invention. For example, currently, adenomyosis is diagnosed after a major surgery (e.g., a hysterectomy). Contrarily, the present invention uses a combination of proteins and metabolites to non-invasively diagnose adenomyosis.

Furthermore, the inventive technical features of the present invention contributed to a surprising result. Adenomyosis is an upper reproductive tract condition and originates in the myometrium (i.e., the middle layer of the uterus), not on the lining. Thus, it was surprising that the present invention was able to use the lower reproductive tract for CVL sampling as an indication of upper tract disease.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, and 1C show immunoregulatory protein levels in the CVL samples were able to distinguish adenomyosis patients from patients with other benign conditions. FIG. 1A shows a volcano plot visualizing the 8 proteins that were significantly up/downregulated in adenomyosis compared to no adenomyosis (p<0.05). FIG. 1B shows scatter plots showing the 3 proteins that were significantly downregulated in adenomyosis, the chemokines IP-10 and GRO, and the cell surface antigen CA19-9. Lines represent the mean. P-values are shown. FIG. 1C shows scatter plots showing the 5 proteins that were significantly upregulated in adenomyosis, the cell surface antigen CEA and cytokines: IL-36γ, TNFβ, IL-13, and IL-9. Line represents the mean. P-values are shown.

FIGS. 2A, 2B, 2C, 2D, 2E, and 2F show global metabolomic profiles that reveal a unique metabolic signature associated with adenomyosis compared to patients with no adenomyosis, made up of mostly amino acids. FIG. 2A shows partial least-squares discriminant analysis comparing patients with adenomyosis to no adenomyosis, showing separation between the 2 groups revealing a unique metabolomic signature belonging to adenomyosis. FIG. 2B shows a bar chart demonstrating the differences in superpathway distribution of metabolites that were detected overall in CVL samples compared to significantly altered metabolites (p<0.05, FC>2) and FDR-corrected significantly altered metabolites (q<0.1 and q<0.05). FIG. 2C shows a volcano plot visualizing the 82 metabolites that were significantly up/downregulated in adenomyosis compared to no adenomyosis (p<0.05, FC>2). 1 significantly downregulated and 81 significantly upregulated. FIG. 2D shows a volcano plot visualizing the 39 metabolites that were significantly upregulated in adenomyosis compared to no adenomyosis and passed FDR-correction (q<0.1, FC>2). FIG. 2E shows a hierarchical clustering analysis heatmap using Pearson clustering and Ward linkage for metabolites and supervised clustering for patient samples—only showing the top 25 most significant metabolites, as determined by T-test. FIG. 2F shows scatter plots highlighting the significant upregulation of key metabolites in adenomyosis with FDR-correction by superpathway (N6-acetyllysine, N-formylmethionine, argininate, pipecolate, 2-hydroxyadipate). Line represents the mean. P- and q-values are shown.

FIGS. 3A, 3B, 3C, and 3D show an enrichment analysis revealed that 32 pathways were significantly enriched in adenomyosis vs. no adenomyosis. FIG. 3A shows an enrichment analysis of adenomyosis patients vs. no adenomyosis patients revealed that 32 pathways were significantly (p<0.05) enriched—top 25 pathways are shown here by superpathway and p-value. Pyrimidine metabolism, carnitine synthesis, and histidine metabolism were significantly enriched pathways. FIG. 3B shows a diagram demonstrating a simplified version of the pyrimidine metabolism pathway and the detection of each metabolite in our analyses. Scatter plots of 3 of the significantly altered metabolites in adenomyosis that demonstrate the enrichment of the pyrimidine metabolism pathway (5,6-dihydrothymine, thymine, N-carbamoylaspartate). Line represents the mean. P- and q-values are shown. FIG. 3C shows a diagram demonstrating a simplified version of the carnitine synthesis pathway and the detection of each metabolite in our analyses. Scatter plots of 3 of the significantly altered metabolites in adenomyosis that demonstrate the enrichment of the carnitine synthesis pathway (N6,N6,N6-trimethyllysine, succinate, deoxycarnitine). Line represents the mean. P- and q-values are shown. FIG. 3D shows a diagram demonstrating a simplified version of the histidine metabolism pathway and the detection of each metabolite in our analyses. Scatter plots of 3 of the significantly altered metabolites in adenomyosis that demonstrate the enrichment of the histidine metabolism pathway (4-imidazoleacetate, formiminoglutamate, histamine). Line represents the mean. P- and q-values are shown. Up arrows indicate metabolites that were significantly upregulated (p<0.05). Bold text represents metabolites shown in scatter plots. Dashed arrows indicate multiple steps in the pathway.

FIG. 4 shows immunoproteomic analysis of cervicovaginal lavage samples revealed pathophysiological processes that may drive the development and/or progression of adenomyotic lesions. Schematic showing the mechanistic links between key immune proteins and metabolites identified in CVL samples and pathophysiological processes that may drive the development and symptomatology of adenomyosis. Triangles represent metabolites/metabolic pathways. Circles represent soluble immune proteins. Dashed arrows represent putative links.

FIGS. 5A, 5B, and 5C shows Metabolomic Multivariate ROC Analysis of CVL samples based on least absolute shrinkage and selection operator (LASSO) selection. Featured biomarkers include N-formylmethionine, X-23423, Argininate, N-carbamoylsarcosine, Fibrinopeptide A, N-methylhydroxyproline, N-methylproline, X-21803. FIG. 5A shows ROC curve, FIG. 5B shows the cross-validation, and FIG. 5C shows the confusion matrix and a table with the performance metrics of the model.

FIGS. 6A, 6B, and 6C shows Immunoproteomics Multivariate ROC Analysis of CVL samples based on least absolute shrinkage and selection operator cl(LASSO) selection. Featured biomarkers included GRO, IL-17A, SHBG, IP-10, CA19-9, TRAIL. FIG. 6A shows ROC curve, FIG. 6B shows the cross-validation, and FIG. 6C shows the confusion matrix and a table with the performance metrics of the model.

FIGS. 7A and 7B shows Metabolomic Multivariate ROC Analysis in vaginal swabs. Featured biomarkers include oxalate, X-11615, X-21803, 3′-dephospho-acetyl-CoA, phenethylamine based on least absolute shrinkage and selection operator cl(LASSO) selection. FIG. 7A shows ROC curve and FIG. 7B shows the cross-validation and a table with the performance metrics of the model.

FIGS. 8A, 8B, and 8C shows Metabolomic and Immunoproteomic Multivariate ROC Analysis of CVL samples based on least absolute shrinkage and selection operator cl(LASSO) selection. Featured biomarkers include N6,N6,N6-trimethyllysine, N-formylmethionine, IL-17A, Argininate, N-carbamoylsarcosine, N-methylhydroxyproline, N-methylproline, N-alpha-acetylornithine, X-21803, 3-formylindole. FIG. 8A shows ROC curve, FIG. 8B shows the cross-validation, and FIG. 8C shows the confusion matrix and a table with the performance metrics of the model.

FIGS. 9A, 9B, and 9C shows Metabolomic Multivariate ROC Analysis of CVL samples. Featured biomarkers include N-formylmethionine, Argininate, N-carbamoylsarcosine, Fibrinopeptide A, N-methylhydroxyproline, N-methylproline based on least absolute shrinkage and selection operator cl(LASSO) selection. FIG. 9A shows ROC curve, FIG. 9B shows the cross-validation, and FIG. 9C shows the confusion matrix and a table with the performance metrics of the model.

FIGS. 10A, 10B, and 10C shows Metabolomic and Immunoproteomic Multivariate ROC Analysis of CVL samples based on least absolute shrinkage and selection operator cl(LASSO) selection. Featured biomarkers include N6,N6,N6-trimethyllysine, N-formylmethionine, IL-17A, Argininate, N-carbamoylsarcosine, N-methylhydroxyproline, N-methylproline, N-alpha-acetylornithine, 3-formylindole. FIG. 10A shows ROC curve, FIG. 10B shows the cross-validation, and FIG. 10C shows the confusion matrix and a table with the performance metrics of the model.

FIGS. 11A, and 11B shows Metabolomic Multivariate ROC Analysis of Vaginal Samples. Featured biomarkers include oxalate, 3′-dephospho-acetyl-CoA, phenethylamine based on least absolute shrinkage and selection operator cl(LASSO) selection. FIG. 11A shows ROC curve, and FIG. 11B shows the cross-validation and a table with the performance metrics of the model.

### DETAILED DESCRIPTION OF THE INVENTION

Following is a list of acronyms as referred to herein:
UMP uridine 5′-monophosphate
SAM S-adenosylmethionine
SAH S-adenosylhomocysteine
NAD nicotinamide adenine dinucleotide
NADH nicotinamide adenine dinucleotide reduced

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human.

As used herein, the terms “normal subject,” “healthy subject,” or “control subject” may be used interchangeably and refers to a subject without adenomyosis.

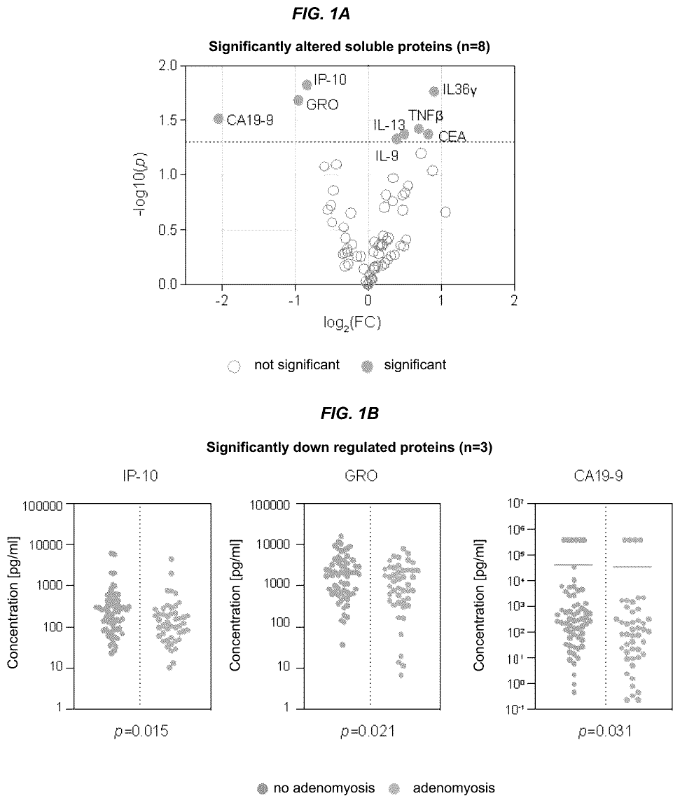

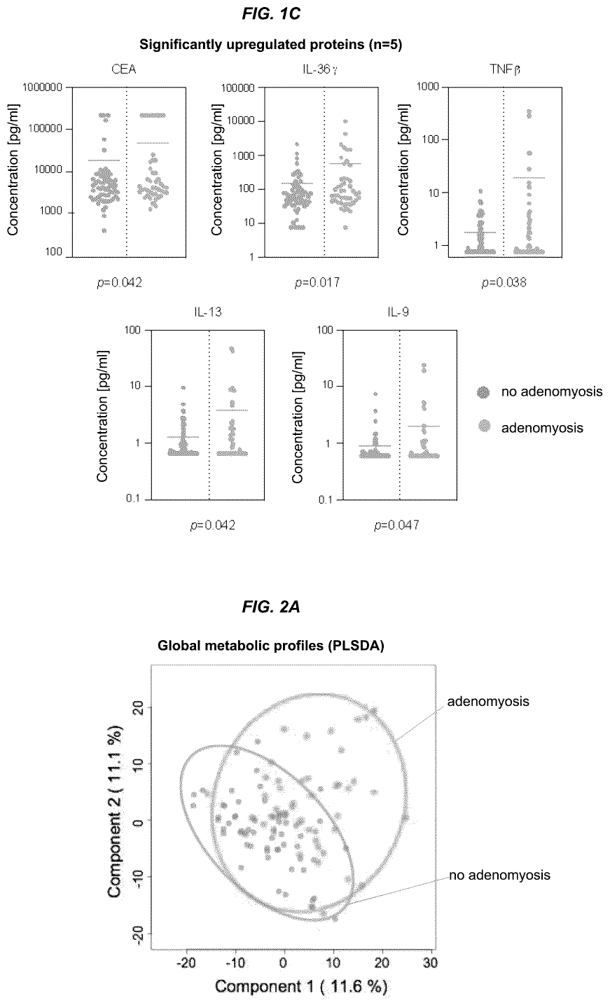

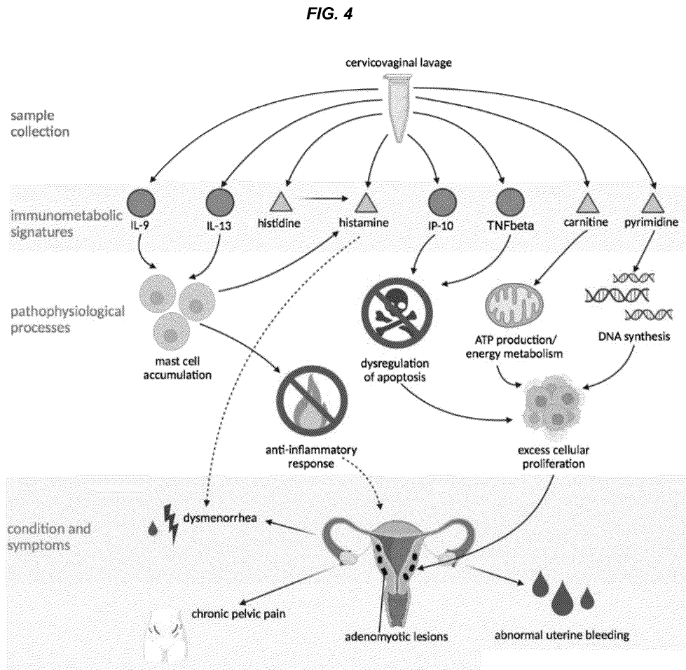

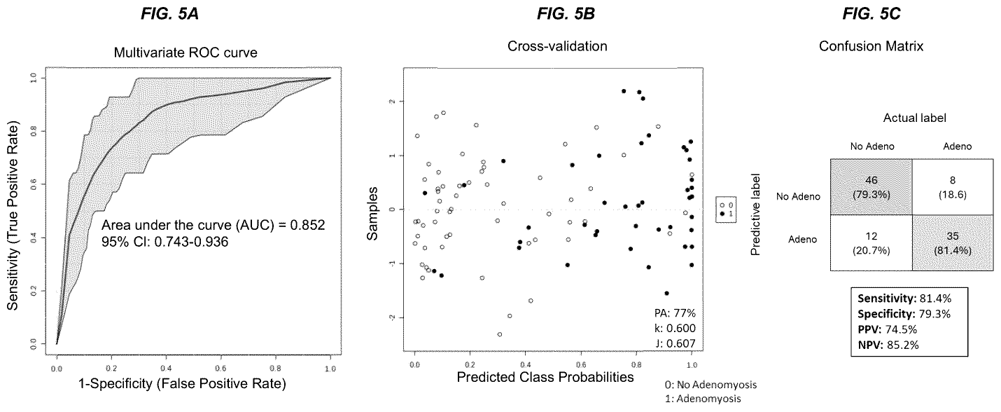

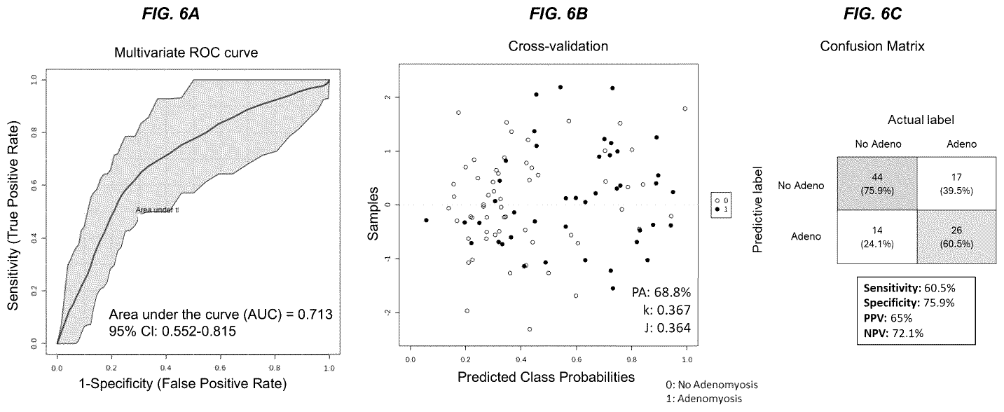

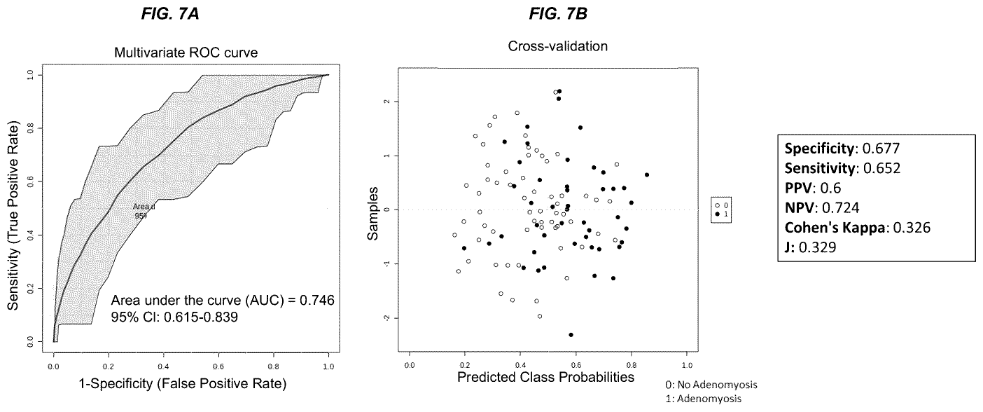

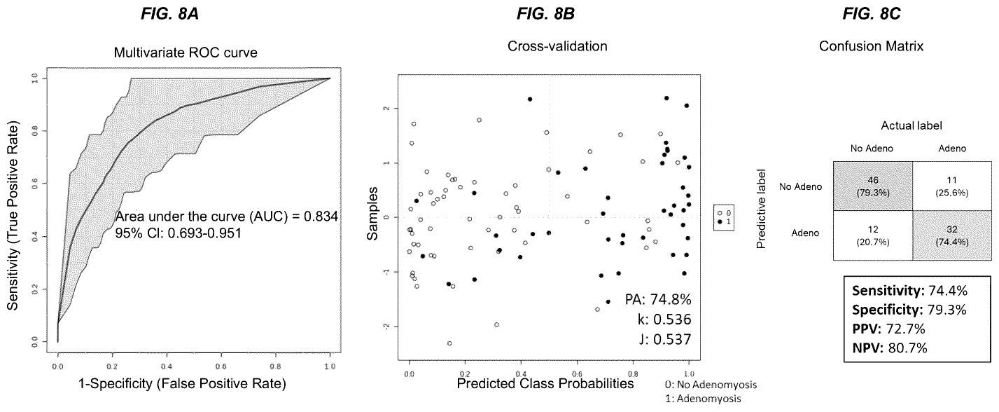

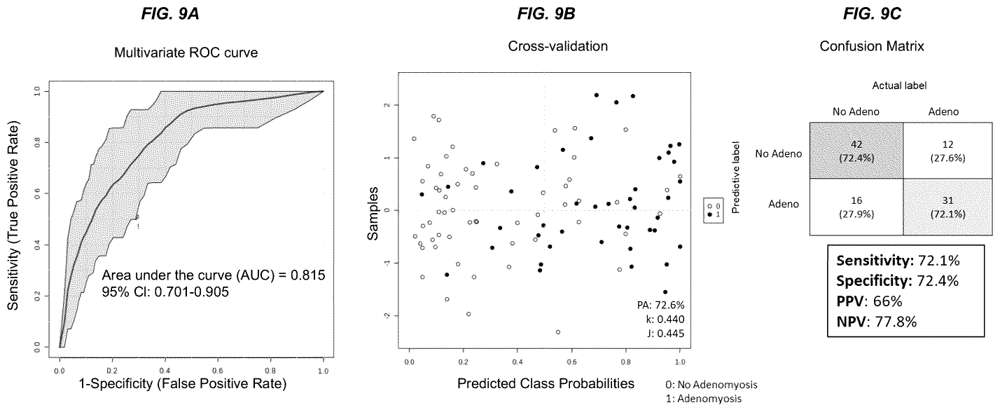

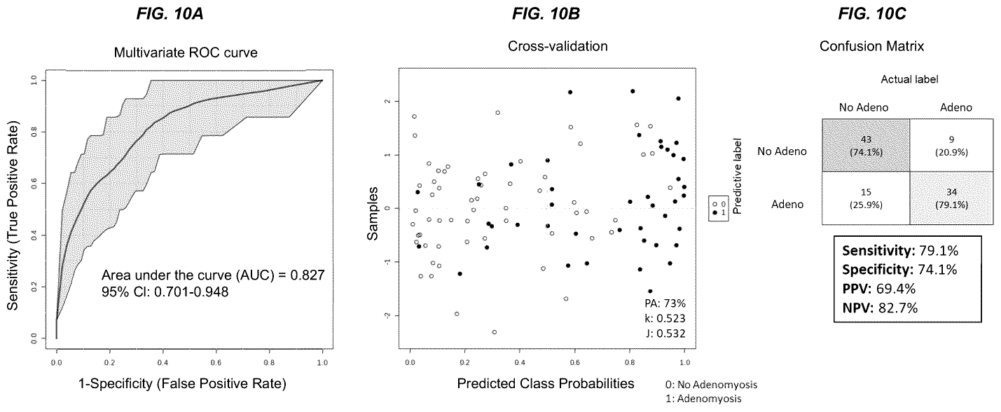

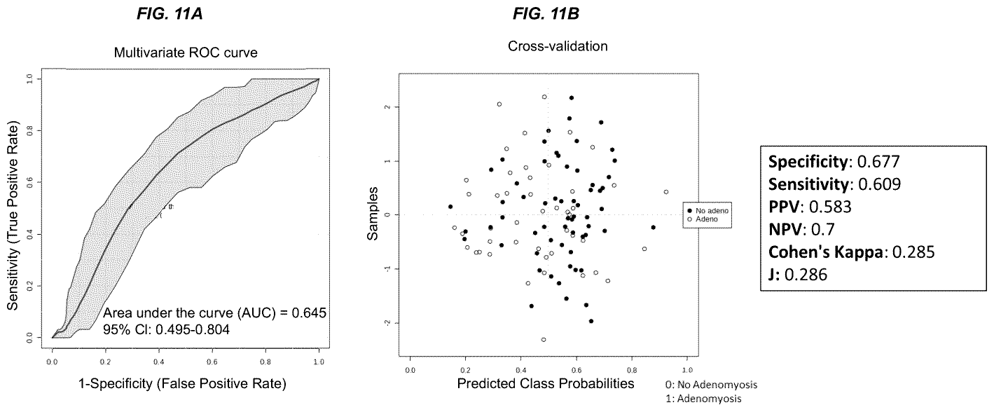

Referring now to FIGS. 1A-11B, the present invention features non-invasive methods for diagnostic screening and early detection of adenomyosis in patients.

The present invention features a non-invasive method of diagnosing a benign gynecologic condition in a patient. In some embodiments, the method comprises determining the patient's levels of five or more biomarkers comprising protein biomarkers, metabolite biomarkers, or a combination thereof. The levels of the five or more biomarkers may be determined by obtaining a biological sample (e.g., a CVL sample, a vaginal swab, or vaginal fluid) from the patient and measuring the levels of five or more biomarkers in the biological sample obtained. The method may further comprise diagnosing the patient with a benign gynecologic condition if the levels of at least five or more biomarkers are altered from a predetermined threshold. In some embodiments, the patient is diagnosed with a benign gynecologic condition if the levels of at least five or more biomarkers are altered from a predetermined threshold. In other embodiments, the patient is diagnosed with the benign gynecologic condition if the levels of at least five or more biomarkers are altered from a control patient (i.e., patients without a benign gynecologic condition). In some embodiments, the benign gynecologic conditions are endometriosis, adenomyosis, fibroids, or a combination thereof. In some embodiments, the method comprises measuring the levels of ten or more biomarkers in the sample obtained. In other embodiments, the method comprises measuring the levels of fifteen or more biomarkers in the sample obtained. In other embodiments, the method comprises measuring the levels of twenty or more biomarkers in the sample obtained.

In some embodiments, the present invention may feature a non-invasive method of diagnosing adenomyosis in a patient. The method may comprise determining the patient's levels of five or more biomarkers comprising protein biomarkers, metabolite biomarkers, or a combination thereof. The levels of the five or more biomarkers may be determined by obtaining a biological sample (e.g., a CVL sample or vaginal swab) from the patient and measuring the levels of five or more biomarkers in the biological sample obtained. The method may further comprise diagnosing the patient with adenomyosis if the patient has levels of at least five or more biomarkers altered from a predetermined threshold. In other embodiments, the patient is diagnosed with adenomyosis if the levels of at least five or more biomarkers are altered from a control patient (i.e., patients without adenomyosis). In some embodiments, the method comprises measuring the levels of ten or more biomarkers in the sample obtained, and thus, the patient may be diagnosed with adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be diagnosed with adenomyosis if the levels of at least fifteen or more biomarkers are altered (e.g., from a predetermined threshold or a control patient). In further embodiments, the method comprises measuring the levels of twenty or more biomarkers in the sample obtained, and thus, the patient may be diagnosed with adenomyosis if the levels of at least twenty or more biomarkers are altered (e.g., from a predetermined threshold or a control patient).

In some embodiments, the present invention features a non-invasive method of diagnosing adenomyosis in a patient. The method may comprise determining the patient's levels of five or more biomarkers comprising protein biomarkers, metabolite biomarkers, or a combination thereof. In some embodiments, the level of five or more biomarkers may be determined by obtaining a cervicovaginal lavage (CVL) sample from the patient and measuring the levels of five or more biomarkers in the CVL sample obtained. In some embodiments, the patient is diagnosed with adenomyosis if the levels of at least five or more biomarkers are altered from a predetermined threshold. In other embodiments, the patient is diagnosed with adenomyosis if the levels of at least five or more biomarkers are altered from a control patient (i.e., patients without adenomyosis). In some embodiments, the method comprises measuring the levels of ten or more biomarkers in the sample obtained, and thus, the patient may be diagnosed with adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be diagnosed with adenomyosis if the levels of at least fifteen or more biomarkers are altered (e.g., from a predetermined threshold or a control patient). In further embodiments, the method comprises measuring the levels of twenty or more biomarkers in the sample obtained, and thus, the patient may be diagnosed with adenomyosis if the levels of at least twenty or more biomarkers are altered (e.g., from a predetermined threshold or a control patient). In some embodiments, the biomarkers may include but are not limited to N-formylmethionine, X-23423, Argininate, N-carbamoylsarcosine, Fibrinopeptide A, N-methylhydroxyproline, N-methylproline, X-21803, GRO, CEA, IL-17A, SHBG, IP-10, IL-9, IL-13, IL-36γ, TNFβ, CA19-9, TRAIL, N6,N6,N6-trimethyllysine, N-alpha-acetylornithine, or 3-formylindole.

In some embodiments, the present invention features a non-invasive method of diagnosing adenomyosis in a patient. The method may comprise determining the patient's levels of five or more biomarkers comprising protein biomarkers, metabolite biomarkers, or a combination thereof. In some embodiments, the level of five or more biomarkers may be determined by obtaining a vaginal swab sample from the patient and measuring the levels of five or more biomarkers in the vaginal swab sample obtained. In some embodiments, the patient is diagnosed with adenomyosis if the levels of at least five or more biomarkers are altered from a predetermined threshold. In other embodiments, the patient is diagnosed with adenomyosis if the levels of at least five or more biomarkers are altered from a control patient (i.e., patients without adenomyosis). In some embodiments, the method comprises measuring the levels of ten or more biomarkers in the sample obtained, and thus, the patient may be diagnosed with adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be diagnosed with adenomyosis if the levels of at least fifteen or more biomarkers are altered (e.g., from a predetermined threshold or a control patient). In further embodiments, the method comprises measuring the levels of twenty or more biomarkers in the sample obtained, and thus, the patient may be diagnosed with adenomyosis if the levels of at least twenty or more biomarkers are altered (e.g., from a predetermined threshold or a control patient). In some embodiments, the biomarkers may include but are not limited to oxalate, X-11615, X-21803, 3′-dephospho-acetyl-CoA, phenethylamine.

In some embodiments, the methods herein comprise determining the levels of five or more biomarkers. In some embodiments, the methods herein comprise determining the levels of six or more biomarkers. In some embodiments, the methods herein comprise determining the levels of seven or more biomarkers. In some embodiments, the methods herein comprise determining the levels of eight or more biomarkers. In some embodiments, the methods herein comprise determining the levels of nine or more biomarkers. In some embodiments, the methods herein comprise determining the levels of ten or more biomarkers. In some embodiments, the methods herein comprise determining the levels of twelve or more biomarkers. In some embodiments, the methods herein comprise determining the levels of 15 or more biomarkers. In some embodiments, the methods herein comprise determining the levels of 20 or more biomarkers. In some embodiments, the methods herein comprise determining the levels of 25 or more biomarkers.

In some embodiments, the methods herein comprise determining the levels of about five biomarkers, about six biomarkers, about seven biomarkers, about eight biomarkers, about nine biomarkers, about ten biomarkers, or about 12 biomarkers, or about 15 biomarkers, or about 20 biomarkers, or about 25 biomarkers.

In some embodiments, the methods herein comprise determining the levels of about 5 to 20 biomarkers, or about 5 to 15 biomarkers, or about 5 to 12 biomarkers, or about 5 to 10 biomarkers, or about 5 to 8 biomarkers. In some embodiments, the methods herein comprise determining the levels of about 8 to 20 biomarkers, or about 8 to 15 biomarkers, or about 8 to 12 biomarkers, or about 8 to 10 biomarkers. In some embodiments, the methods herein comprise determining the levels of about 10 to 20 biomarkers, or about 10 to 15 biomarkers, or about 10 to 12 biomarkers.

In some embodiments, the level of a biomarker may refer to its relative abundance or intensity of said biomarker. As used herein, “relative abundance” may refer to the level of a biomarker being measured in a patient compared to the level of a biomarker being measured in control patients (i.e., patients without a benign gynecologic condition, e.g., adenomyosis). In accordance with the present invention, methods well known in the art (e.g., immunoassays or liquid chromatography-mass spectrometry or the like) may be used to measure the levels of the biomarkers.

In some embodiments, the patient is diagnosed with a benign gynecologic condition (e.g., endometriosis, adenomyosis, fibroids, or a combination thereof) if the levels of at least five or more biomarkers, or at least six or more biomarkers, or at least seven or more biomarkers, or at least eight or more biomarkers, or at least nine or more biomarkers, or at least ten or more biomarkers, or at least twelve or more biomarkers, or at least 15 or more biomarkers, or at least 20 or more biomarkers, or at least 25 or more biomarkers are altered (e.g., from a predetermined threshold or a control patient). In some embodiments, the patient is diagnosed with a benign gynecologic condition (e.g., endometriosis, adenomyosis, fibroids, or a combination thereof) if the levels of about five biomarkers, about six biomarkers, about seven biomarkers, about eight biomarkers, about nine biomarkers, about ten biomarkers, or about twelve biomarkers, or about 15 biomarkers, or about 20 biomarkers, or about 25 biomarkers are altered (e.g., from a predetermined threshold or a control patient).

In some embodiments, the patient is diagnosed with adenomyosis if the levels of at least five or more biomarkers are altered (e.g., from a predetermined threshold or a control patient). In some embodiments, the patient is diagnosed with adenomyosis if the levels of at least six or more biomarkers are altered (e.g., from a predetermined threshold or a control patient). In some embodiments, the patient is diagnosed with adenomyosis if the levels of at least seven or more biomarkers are altered (e.g., from a predetermined threshold or a control patient). In some embodiments, the patient is diagnosed with adenomyosis if the levels of at least eight or more biomarkers are altered (e.g., from a predetermined threshold or a control patient). In some embodiments, the patient is diagnosed with adenomyosis if the levels of at least nine or more biomarkers are altered (e.g., from a predetermined threshold or a control patient). In some embodiments, the patient is diagnosed with adenomyosis if the levels of at least ten or more biomarkers are altered (e.g., from a predetermined threshold or a control patient). In some embodiments, the patient is diagnosed with adenomyosis if the levels of at least twelve or more biomarkers are altered (e.g., from a predetermined threshold or a control patient). In some embodiments, the patient is diagnosed with adenomyosis if the levels of at least 15 or more biomarkers are altered (e.g., from a predetermined threshold or a control patient). In some embodiments, the patient is diagnosed with adenomyosis if the levels of at least 20 or more biomarkers are altered (e.g., from a predetermined threshold or a control patient). In some embodiments, the patient is diagnosed with adenomyosis if the levels of at least 25 or more biomarkers are altered (e.g., from a predetermined threshold or a control patient).

In some embodiments, the patient is diagnosed with adenomyosis if the levels of about five biomarkers, or about six biomarkers, or about seven biomarkers, or about eight biomarkers, or about nine biomarkers, about ten biomarkers, or about twelve biomarkers, or about 15 biomarkers, or about 20 biomarkers, or about 25 biomarkers are altered (e.g., from a predetermined threshold or a control patient).

In certain embodiments, the described methods may involve assessing an extensive range of biomarkers beyond the necessary requirements for diagnosing adenomyosis in a patient. For instance, methods herein may comprise evaluating about 25 biomarkers, whereby a diagnosis of adenomyosis may be established if at least 10 biomarkers are altered (e.g., from a predetermined threshold or a control patient).

In some embodiments, the protein biomarkers may comprise cell surface antigen biomarkers, chemokine biomarkers, cytokine biomarkers, or a combination thereof. In some embodiments, the cell surface antigen biomarkers comprise carbohydrate antigen 19-9 (CA19-9), carcinoembryonic antigen (CEA), or a combination thereof. In some embodiments, the chemokine biomarkers comprise growth regulated oncogene (GRO), interferon γ-induced protein 10 kDa (IP-10), or a combination thereof. In some embodiments, the cytokine biomarkers comprise Interleukin (IL)-9, IL-13, IL-36γ, tumor necrosis factor-beta (TNFβ), or a combination thereof.

In certain embodiments, the protein biomarkers may comprise CA19-9, CEA, GRO, IP-10, IL-9, IL-13, IL-36γ, TNFβ, or a combination thereof. In some embodiments, the protein biomarkers comprise IL36γ, epidermal growth factor (EGF), fibroblast growth factor 2 (FGF-2), eotaxin, transforming growth factor alpha (TGF-α), granulocyte colony stimulating factor (G-CSF), FMS-like tyrosine kinase 3 ligan (Flt-3L), granulocyte-macrophage colony-stimulating factor (GM-CSF), Fractalkine, interferon alpha-2 (IFNα2), IFNγ, growth regulated oncogene (GRO), IL-10, monocyte chemotactic protein 3 (MCP-3), p40 subunit of IL-12 (IL-12p40), macrophage-derived chemokine (MDC), Interleukin-12, p70 (IL-12 p70), platelet-derived growth factor AA (PDGF-AA), IL-13, PDGF-AB/BB, IL-15, soluble CD40 ligand (sCD40L), IL-17A, IL-1α, IL-9, IL-1B, IL-2, IL-4, IL-5, IL-6, IL-7, IL-8, interferon gamma-induced protein 10 (IP-10), monocyte chemoattractant protein-1 (MCP-1/CCL2), macrophage inflammatory protein-1 alpha (MIP-1α/CCL3), MIP-1β, RANTES (Regulated upon Activation, Normal T Cell Expressed and Presumably Secreted/CCL5), tumor necrosis factor-beta (TNFβ), TNFα, alpha fetoprotein (AFP), prostate-specific antigen (PSA), Cancer antigen 15-3 (CA15-3), CA19-9, macrophage migration inhibitory factor (MIF), TNF-related apoptosis-inducing ligand (TRAIL), Leptin, soluble Fas ligand (sFasL), CEA, CA125, hepatocyte growth factor (HGF), sFas, prolactin, SKP1, CUL1, F-box protein (SCF) complex, cytokeratin-19 fragments (CYFRA 21-1), osteopontin (OPN), human epididymis protein 4 (HE4), vascular endothelial growth factor (VEGF), B and T lymphocyte attenuator (BTLA), CD27, CD28, T cell immunoglobulin and mucin domain-containing protein 3 (TIM3), herpesvirus entry mediator (HVEM), CD40, lymphocyte-activation gene 3 (LAG-3), toll-like receptor 2 (TLR-2), glucocorticoid-induced TNFR-related protein (GITR) ligand (GITRL), programmed cell death protein 1 (PD-1), CD80, CD86, programmed death-ligand 1 (PD-L1), PD-L2, ICOS (Inducible T Cell Costimulator), or a combination thereof.

In some embodiments, the cell surface antigen biomarker CA19-9 is downregulated in patients diagnosed with adenomyosis compared to patients without adenomyosis. In some embodiments, the cell surface antigen biomarker CEA is upregulated in patients diagnosed with adenomyosis compared to patients without adenomyosis. In some embodiments, the chemokine biomarkers (e.g., growth regulated oncogene (GRO), interferon γ-induced protein 10 kDa (IP-10)) are downregulated in patients diagnosed with adenomyosis compared to patients without adenomyosis. In some embodiments, the cytokine biomarkers (e.g., Interleukin (IL)-9, IL-13, IL-36γ, tumor necrosis factor-beta (TNFβ)) are upregulated in patients diagnosed with adenomyosis compared to patients without adenomyosis.

In some embodiments, the metabolite biomarkers may comprise amino acid biomarkers, lipid biomarkers, xenobiotics, or a combination thereof. In some embodiments, the amino acid biomarkers comprise N6-acetyllysine, N-formylmethionine, argininate, pipecolate, or a combination thereof. In some embodiments, the lipid biomarker comprises 2-hydroxyadipate.

In certain embodiments, the metabolite biomarkers comprise N6-acetyllysine, N-formylmethionine, argininate, pipecolate, 2-hydroxyadipate, or a combination thereof. In some embodiments, the metabolite biomarkers comprise N6,N6,N6-trimethyllysine, N-methylhydroxyproline, N6-acetyllysine, N-formylmethionine, argininate, X-23423, 2-hydroxyadipate, 5,6-dihydrothymine, pipecolate, sarcosine, dihydroorotate, N-alpha-acetylornithine, N-formylphenylalanine, N-acetylleucine, thymine, gamma-glutamyl-epsilon-lysine, 3-formylindole, succinate, X-23908, 4-imidazoleacetate, N-methylproline, X-25958, N-carbamoylaspartate, isobutyrylglycine (C4), X-25047, N6,N6-dimethyllysine, deoxycarnitine, 2-oxoarginine, N-acetyl-2-aminoadipate, formiminoglutamate, 2-hydroxy-4-(methylthio)butanoic acid, N-acetylmethionine, 2-hydroxyglutarate, N-acetylglutamate, X-24724, methyl-4-hydroxybenzoate sulfate, N-carbamoylsarcosine, 4-hydroxyphenylacetate, 3-(4-hydroxyphenyl) propionate, N-acetyltaurine, 2-isopropylmalate, 1-methylhistamine, alpha-hydroxyisocaproate, citraconate/glutaconate, Fibrinopeptide A, phenethylamine, alpha-hydroxyisovalerate, N-acetyl-cadaverine, N2-acetyl,N6,N6-dimethyllysine, X-23738, serotonin, pantoate, X-19913, 2-piperidinone, N,N-dimethyl-pro-pro, 3-phenylpropionate (hydrocinnamate), diacetylspermidine, Fibrinopeptide A (3-16)**, N-acetylhistamine, X-21796, orotidine, tyramine, tryptamine, X-25810, 4-acetamidophenol, 5-aminovalerate, N-acetylaspartate (NAA), Fibrinopeptide A, des-ala(1), imidazole propionate, histamine, 1-methyl-5-imidazolelactate, X-24410, stachydrine, 2-keto-3-deoxy-gluconate, margaroylcarnitine (C17), 1-palmitoyl-2-linoleoyl-GPE (16:0/18:2), dopamine 3-O-sulfate, indolepropionate, N-acetylputrescine, X-21803, 2-hydroxy-3-methylvalerate, or a combination thereof.

In some embodiments, the amino acid biomarkers (e.g., N6-acetyllysine, N-formylmethionine, argininate, pipecolate) are upregulated in patients diagnosed with adenomyosis compared to patients without adenomyosis. In some embodiments, the lipid biomarker (e.g., 2-hydroxyadipic) is upregulated in patients diagnosed with adenomyosis compared to patients without adenomyosis.

The present invention may further comprise a method comprising obtaining a biological sample (e.g., a CVL sample or vaginal swab, or vaginal fluid) from a patient, producing a profile of the biological sample previously collected by detecting at least five or more biomarkers comprising protein biomarkers, metabolite biomarkers or a combination thereof, and analyzing the biological sample profile produced.

In some embodiments, the present invention may further comprise a method comprising obtaining a cervicovaginal lavage (CVL) sample from a patient, producing a profile of the CVL sample previously collected by detecting at least five or more biomarkers comprising proteins and metabolite and analyzing the CVL sample profile produced. In some embodiments, the biomarkers may include but are not limited to N-formylmethionine, X-23423, Argininate, N-carbamoylsarcosine, Fibrinopeptide A, N-methylhydroxyproline, N-methylproline, X-21803, GRO, CEA, IL-17A, SHBG, IP-10, IL-9, IL-13, IL-36γ, TNFβ, CA19-9, TRAIL, N6,N6,N6-trimethyllysine, N-alpha-acetylornithine, or 3-formylindole. The method may further comprise centrifuging the CVL sample to remove mucus and cellular debris. Without wishing to limit the present invention to any theory or mechanism, it is believed that centrifuging the CVL sample gives a cleaner (e.g., decreases cellular debris) and more soluble fluid to work with when producing a profile (e.g., using fluidics and microfluidics systems). In some embodiments, the samples described herein require minimal to no process to allow for the detection of the biomarkers.

In other embodiments, the present invention may further comprise a method comprising obtaining a vaginal swab sample from a patient, producing a profile of the vaginal swab sample previously collected by detecting at least five or more biomarkers comprising proteins and metabolite and analyzing the CVL sample profile produced. In some embodiments, the biomarkers may include but are not limited to oxalate, X-11615, X-21803, 3′-dephospho-acetyl-CoA, phenethylamine.

The present invention may feature a method of treating a benign gynecologic condition in a patient in need thereof. The method may comprise diagnosing the patient with a benign gynecologic condition by obtaining a biological sample (e.g., a CVL sample, a vaginal swab, or vaginal fluid) from the patient and measuring the levels of five or more biomarkers in the biological sample obtained. In some embodiments, the patient is diagnosed with a benign gynecologic condition if the levels of at least five or more biomarkers are altered from a predetermined threshold. In other embodiments, the patient is diagnosed with the benign gynecologic condition if the levels of at least ten or more biomarkers are altered from a control patient (i.e., patients without a benign gynecologic condition). The method may further comprise administering a therapeutic amount of a treatment to the patient if the patient is diagnosed with a benign gynecologic condition. In some embodiments, the benign gynecologic conditions are endometriosis, adenomyosis, fibroids, or a combination thereof.

In some embodiments, the present invention features a method of treating adenomyosis in a patient in need thereof. The method may comprise diagnosing the patient with adenomyosis by obtaining a biological sample (e.g., a CVL sample or a vaginal swab) from the patient and measuring the levels of five or more biomarkers in the sample obtained. In some embodiments, the patient is diagnosed with adenomyosis if the levels of at least five or more biomarkers are altered from a predetermined threshold. In other embodiments, the patient is diagnosed with adenomyosis if the levels of at least ten or more biomarkers are altered from a control patient (i.e., patients without adenomyosis). In some embodiments, the method comprises measuring the levels of ten or more biomarkers in the sample obtained, and thus, the patient may be diagnosed with adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be diagnosed with adenomyosis if the levels of at least fifteen or more biomarkers are altered (e.g., from a predetermined threshold or a control patient). The method may further comprise administering a therapeutic amount of a treatment to the patient if the patient is diagnosed with adenomyosis.

The present invention may also feature methods of treating adenomyosis in a patient in need thereof. The method may comprise diagnosing the patient with adenomyosis as described herein. For example, diagnosing adenomyosis may comprise obtaining a cervicovaginal lavage (CVL) sample from the patient and measuring the levels of five or more biomarkers in the sample obtained. In some embodiments, the patient is diagnosed with adenomyosis if the levels of at least five or more biomarkers are altered from a predetermined threshold. In other embodiments, the patient is diagnosed with adenomyosis if the levels of at least ten or more biomarkers are altered from a control patient (i.e., patients without adenomyosis). In some embodiments, the method comprises measuring the levels of ten or more biomarkers in the sample obtained, and thus, the patient may be diagnosed with adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be diagnosed with adenomyosis if the levels of at least fifteen or more biomarkers are altered (e.g., from a predetermined threshold or a control patient). The method may further comprise administering a therapeutic amount of a treatment to the patient if the patient is diagnosed with adenomyosis. In some embodiments, the biomarkers may include but are not limited to N-formylmethionine, X-23423, Argininate, N-carbamoylsarcosine, Fibrinopeptide A, N-methylhydroxyproline, N-methylproline, X-21803, GRO, CEA, IL-17A, SHBG, IP-10, IL-9, IL-13, IL-36γ, TNFβ, CA19-9, TRAIL, N6,N6,N6-trimethyllysine, N-alpha-acetylornithine, or 3-formylindole.

In some embodiments, the present invention features a method of treating adenomyosis in a patient in need thereof. The method may comprise diagnosing the patient with adenomyosis by obtaining a vaginal swab sample from the patient and measuring the levels of five or more biomarkers in the sample obtained. In some embodiments, the patient is diagnosed with adenomyosis if the levels of at least five or more biomarkers are altered from a predetermined threshold. In some embodiments, the method comprises measuring the levels of ten or more biomarkers in the sample obtained, and thus, the patient may be diagnosed with adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be diagnosed with adenomyosis if the levels of at least fifteen or more biomarkers are altered (e.g., from a predetermined threshold or a control patient). In some embodiments, the biomarkers may include but are not limited to oxalate, X-11615, X-21803, 3′-dephospho-acetyl-CoA, phenethylamine. The method may further comprise administering a therapeutic amount of a treatment to the patient if the patient is diagnosed with adenomyosis

In some embodiments, the five or more biomarkers comprise protein biomarkers, metabolite biomarkers, or a combination thereof. Non-limiting examples of the biomarkers include carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, tumor necrosis factor-beta (TNFβ), N6-acetyllysine, N-formylmethionine, argininate, pipecolate, 2-hydroxyadipic or a combination thereof.

Treatments that may be used in accordance with the present invention may include either nonsurgical treatment such as contraceptives (e.g., hormonal contraceptives; e.g., oral contraceptives) and other combination hormonal therapies; or surgical procedures such as a hysterectomy.

In some embodiments, the biological sample may comprise cervicovaginal lavage (CVL) sample, urine, vaginal swab or vaginal fluid, or other secretions (e.g., menstrual fluid collected from a menstrual cup or other devices), a cervicovaginal lavage (CVL) sample, a urine sample, a vaginal swab or vaginal fluid, or a cervicovaginal secretion (e.g., a cervicovaginal secretion that is collected via a self-collected lavage or a menstrual cup).

The present invention may also feature a method of distinguishing between adenomyosis and endometriosis in a subject. The method may comprise obtaining a biological sample (e.g., from the subject; e.g., a CVL sample, a vaginal swab, or vaginal fluid) and measuring the levels of five or more biomarkers in the sample obtained. In some embodiments, the method comprises obtaining a cervicovaginal lavage (CVL) sample and measuring the levels of five or more biomarkers in the sample obtained. In other embodiments, the method comprises obtaining a vaginal swab sample and measuring the levels of five or more biomarkers in the sample obtained. In some embodiments, the patient is determined to have adenomyosis if the levels of at least five or more biomarkers are altered from a predetermined threshold. In other embodiments, the patient is determined to have adenomyosis if the levels of at least five or more biomarkers are altered from a control patient (i.e., patients without adenomyosis). In some embodiments, the method comprises measuring the levels of ten or more biomarkers in the sample obtained, and thus, the patient may be determined to have adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be determined to have adenomyosis if the levels of at least fifteen or more biomarkers are altered (e.g., from a predetermined threshold or a control patient). In some embodiments, the biomarkers comprise protein biomarkers, metabolite biomarkers, or a combination thereof. In certain embodiments, such as when analyzing a CVL sample, the biomarkers may comprise N-formylmethionine, X-23423, Argininate, N-carbamoylsarcosine, Fibrinopeptide A, N-methylhydroxyproline, N-methylproline, X-21803, GRO, CEA, IL-17A, SHBG, IP-10, IL-9, IL-13, IL-36γ, TNFβ, CA19-9, TRAIL, N6,N6,N6-trimethyllysine, N-alpha-acetylornithine, or 3-formylindole. In other embodiments, such as when analyzing a vaginal sample, the biomarkers may comprise oxalate, X-11615, X-21803, 3′-dephospho-acetyl-CoA, phenethylamine.

The present invention may further feature an in vitro method of diagnosing a benign gynecologic condition (e.g., endometriosis, adenomyosis, fibroids, or a combination thereof) in a subject in need thereof. The method may comprise producing a profile from a biological sample (e.g., a CVL sample, a vaginal swab, or vaginal fluid) obtained from the subject by detecting at least five or more biomarkers and analyzing the biological sample profile produced. In some embodiments, the patient is diagnosed with a benign gynecologic condition if the levels of at least five or more biomarkers are altered from a predetermined threshold. In other embodiments, the patient is diagnosed with the benign gynecologic condition if the levels of at least five or more biomarkers are altered from a control patient (i.e., patients without a benign gynecologic condition).

In some embodiments, the present invention features an in vitro method of diagnosing adenomyosis in a subject in need thereof. The method may comprise producing a profile from a biological sample (e.g., a CVL sample, a vaginal swab, or vaginal fluid) obtained from the subject by detecting at least five or more biomarkers and analyzing the biological sample profile produced. In some embodiments, the patient is diagnosed with adenomyosis if the levels of at least five or more biomarkers are altered from a predetermined threshold. In other embodiments, the patient is diagnosed with adenomyosis if the levels of at least five or more biomarkers are altered from a control patient (i.e., patients without adenomyosis). In some embodiments, the method comprises measuring the levels of ten or more biomarkers in the sample obtained, and thus, the patient may be diagnosed with adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be diagnosed with adenomyosis if the levels of at least fifteen or more biomarkers are altered (e.g., from a predetermined threshold or a control patient). In some embodiments, the biomarkers comprise protein biomarkers, metabolite biomarkers, or a combination thereof.

In some embodiments, the aforementioned method (e.g., the in vitro method of diagnosing adenomyosis) comprises producing a profile from a cervicovaginal lavage (CVL) sample obtained from the subject by detecting at least five or more biomarkers and analyzing the CVL sample profile produced. In other embodiments, the aforementioned method (e.g., the in vitro method of diagnosing adenomyosis) comprises producing a profile from a vaginal swab sample obtained from the subject by detecting at least five or more biomarkers and analyzing the vaginal swab sample profile produced. In some embodiments, the patient is diagnosed with adenomyosis if the levels of at least five or more biomarkers are altered from a predetermined threshold. In other embodiments, the patient is diagnosed with adenomyosis if the levels of at least five or more biomarkers are altered from a control patient (i.e., patients without adenomyosis). In some embodiments, the method comprises measuring the levels of ten or more biomarkers in the sample obtained, and thus, the patient may be diagnosed with adenomyosis if the levels of at least ten or more biomarkers are altered (e.g., from a predetermined threshold or a control patient). In other embodiments, the method comprises measuring the levels of fifteen or more biomarkers in the sample obtained, and thus, the patient may be diagnosed with adenomyosis if the levels of at least fifteen or more biomarkers are altered (e.g., from a predetermined threshold or a control patient). In some embodiments, the biomarkers comprise protein biomarkers, metabolite biomarkers, or a combination thereof. In certain embodiments, such as when analyzing a CVL sample, the biomarkers may comprise N-formylmethionine, X-23423, Argininate, N-carbamoylsarcosine, Fibrinopeptide A, N-methylhydroxyproline, N-methylproline, X-21803, GRO, CEA, IL-17A, SHBG, IP-10, IL-9, IL-13, IL-36γ, TNFβ, CA19-9, TRAIL, N6,N6,N6-trimethyllysine, N-alpha-acetylornithine, or 3-formylindole. In other embodiments, such as when analyzing a vaginal sample, the biomarkers may comprise oxalate, X-11615, X-21803, 3′-dephospho-acetyl-CoA, phenethylamine.

In some embodiments, the biomarkers comprise protein biomarkers, metabolite biomarkers, or a combination thereof. Non-limiting examples of the biomarkers include carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, tumor necrosis factor-beta (TNFβ), N6-acetyllysine, N-formylmethionine, argininate, pipecolate, 2-hydroxyadipic or a combination thereof.

### EXAMPLE 1

The following is a non-limiting example of the present invention. It is to be understood that said example is not intended to limit the present invention in any way. Equivalents or substitutes are within the scope of the present invention.

Participants and sample collection: One hundred and eight participants undergoing hysterectomy for benign conditions were recruited at two clinical sites in the Phoenix (AZ, USA) metropolitan area: Banner University Medical Center—Phoenix. Histopathology of biopsy samples collected from the surgery was used for the stratification of participants into two groups: adenomyosis (n=46) and no adenomyosis (n=62). Women were excluded from the study if they were currently menstruating; currently lactating; currently on antibiotics, antifungals, antivirals, or topical steroids; currently (or within the past 3 months) had vaginal, vulvar, urinary tract or sexually transmitted infections; used any douching products, vaginal medications or suppositories, feminine deodorant sprays, wipes, or lubricants within the past 48 hours; used any depilatory treatments in the genital area in the past 72 hours; had any skin condition in the genital area; had sexual intercourse in the past 48 hours; were bathing or swimming in the past 4 hours; were smoking or consuming nicotine-containing products in the past 2 hours; had diabetes or hepatitis; were HIV-positive. Inclusion criteria included any women, 18 years of age and older, of any race and ethnicity, who were undergoing hysterectomy for benign conditions. Demographic, socioeconomic, and medical history data were collected from surveys and/or medical records (Table 1).

TABLE 1
Patients demographics show no significant difference in demographic, socioeconomic,
or medical history between adenomyosis and no adenomyosis patients. P-
values were calculated using the Wilcoxon rank sum test for continuous
variables and Fisher's exact test for categorical variables.

| | All | | Adenomyosis | | No Adenomyosis | | |
|---|---|---|---|---|---|---|---|
| | (n = 108) | | (n = 46) | | (n = 62) | | p-value |
| Age (mean (S.D)) | 45.55 | (10.01) | 45.52 | (8.92) | 45.58 | (10.83) | 0.65 |
| Race (n = 107) | | | | | | | 0.73 |
| American Indian/ | 5 | (4.67%) | 2 | (4.92%) | 3 | (4.92%) | |
| Alaska Native | | | | | | | |
| White/Caucasian | 78 | (72.90%) | 34 | (73.91%) | 44 | (72.13%) | |
| Black or African American | 11 | (10.28%) | 6 | (13.04%) | 5 | (8.20%) | |
| All Other | 13 | (12.15%) | 4 | (8.70%) | 9 | (14.75%) | |
| Ethnicity (n = 108) | | | | | | | 0.52 |
| Non-Hispanic | 76 | (70.37%) | 34 | (73.91%) | 42 | (67.74%) | |
| Hispanic | 32 | (29.32%) | 12 | (26.09%) | 20 | (32.26%) | |
| BMI (mean (S.D)) (n = 108) | 30.63 | (7.55%) | 30.36 | (6.58%) | 30.83 | (8.23%) | 0.89 |
| BMI (n = 108) | | | | | | | 0.12 |
| <25 | 23 | (21.30%) | 6 | (13.04%) | 17 | (27.42%) | |
| 25-29 | 38 | (35.19%) | 21 | (45.65%) | 17 | (27.42%) | |
| 30-34 | 19 | (17.59%) | 9 | (19.57%) | 10 | (16.13%) | |
| ≥35 | 28 | (25.93%) | 10 | (21.74%) | 18 | (29.03%) | |
| Education (n = 105) | | | | | | | 0.20 |
| Less than high school | 3 | (2.86%) | 0 | (0.00%) | 3 | (5.08%) | |
| High school diploma or GED | 20 | (19.05%) | 11 | (23.91%) | 9 | (15.25%) | |
| Some college | 24 | (22.86%) | 14 | (30.43%) | 10 | (16.95%) | |
| Association degree or | 22 | (20.95%) | 8 | (17.39%) | 14 | (23.73%) | |
| technical certificate | | | | | | | |
| Bachelor degree | 22 | (20.95%) | 9 | (19.57%) | 13 | (22.03%) | |
| Master/Doctor degree | 14 | (13.33%) | 4 | (8.70%) | 10 | (16.95%) | |
| Income (n = 99) | | | | | | | 0.43 |
| <10,000 | 3 | (3.03%) | 2 | (4.35%) | 2 | (4.35%) | |
| 10,000-25,000 | 10 | (10.10%) | 7 | (15.22%) | 7 | (15.22%) | |
| 25,000-50,000 | 13 | (13.13%) | 5 | (10.87%) | 5 | (10.87%) | |
| 50,000-75,000 | 23 | (23.23%) | 10 | (21.74%) | 10 | (21.74%) | |
| 75,000-100,000 | 15 | (15.15%) | 9 | (19.57%) | 6 | (11.32%) | |
| >100,000 | 24 | (24.24%) | 8 | (17.39%) | 16 | (30.19%) | |
| Don't know/refused | 11 | (11.11%) | 5 | (10.97%) | 6 | (11.32%) | |
| Employment status (n-103) | | | | | | | 0.65 |
| Yes | 75 | (72.82%) | 34 | (75.56%) | 41 | (70.69%) | |
| No | 28 | (27.18%) | 11 | (24.44%) | 17 | (29.31%) | |
| Marital Status (n = 108) | | | | | | | 0.12 |
| Single/Divorced/Widowed | 41 | (37.96%) | 21 | (45.65%) | 20 | (32.26%) | |
| Married | 60 | (55.56%) | 22 | (47.83%) | 38 | (61.29%) | |
| Cohabitating | 5 | (4.63%) | 1 | (2.17%) | 4 | (6.45%) | |
| Other | 1 | (1.85%) | 2 | (4.35%) | 0 | (0.00%) | |
| Sex Orientation (n = 100) | | | | | | | 0.99 |
| Heterosexual | 93 | (93.00%) | 41 | (93.18%) | 52 | (92.86%) | |
| Bisexual | 2 | (2.00%) | 1 | (2.27%) | 1 | (1.79%) | |
| Homosexual | 5 | (5.00%) | 2 | (4.55%) | 3 | (5.36%) | |
| Alcohol use (n = 101) | | | | | | | 0.72 |
| Yes | 50 | (49.50%) | 23 | (53.49%) | 27 | (46.55%) | |
| No | 47 | (46.53%) | 19 | (44.19%) | 28 | (48.28%) | |
| Quit | 4 | (3.96%) | 1 | (2.33%) | 3 | (5.17%) | |
| Tobacco use (n = 104) | | | | | | | 0.13 |
| Yes | 14 | (13.46%) | 7 | (15.56%) | 7 | (11.86%) | |
| No | 34 | (32.69%) | 18 | (40.00%) | 16 | (27.12%) | |
| Never | 47 | (45.19%) | 19 | (42.22%) | 28 | (47.46%) | |
| Quit | 9 | (8.65%) | 1 | (2.22%) | 8 | (13.56%) | |
| Douching (n = 94) | | | | | | | 0.99 |
| Yes | 15 | (15.96%) | 7 | (17.07%) | 8 | (15.38%) | |
| No | 79 | (84.04%) | 34 | (82.93%) | 45 | (84.91%) | |
| Menopausal status (n = 108) | | | | | | | 0.62 |
| Pre | 89 | (82.41%) | 39 | (84.65%) | 50 | (80.65%) | |
| Post | 19 | (17.59%) | 7 | (15.22%) | 12 | (19.35%) | |
| Previous dilation and | | | | | | | 0.80 |
| curettage (n = 108) | | | | | | | |
| Yes | 19 | (17.59%) | 9 | (19.57%) | 10 | (16.13%) | |
| No | 89 | (82.43%) | 37 | (80.43%) | 52 | (83.87%) | |
| Co-occurring conditions | | | | | | | |
| (n = 108) | | | | | | | |
| Endometriosis | 21 | (19.44%) | 11 | (23.09%) | 10 | (16.13%) | 0.31 |
| Fibroids | 70 | (64.81%) | 30 | (65.22%) | 40 | (64.52%) | 0.94 |
| Parity (n = 107) | | | | | | | 0.09 |
| 0 | 22 | (20.56%) | 7 | (15.22%) | 15 | (24.59%) | |
| 1 | 9 | (8.41%) | 2 | (4.35%) | 7 | (11.48%) | |
| 2 | 23 | (21.50%) | 9 | (19.57%) | 14 | (22.95%) | |
| 3 | 27 | (25.23%) | 11 | (23.91%) | 16 | (26.23%) | |
| 4+ | 26 | (24.30%) | 17 | (36.96%) | 9 | (14.75%) | |
| Heavy periods (n = 94) | | | | | | | 0.61 |
| Light | 5 | (5.32%) | 3 | (6.82%) | 2 | (4.00%) | |
| Mod | 21 | (22.34%) | 8 | (18.18%) | 13 | (26.00%) | |
| Heavy | 68 | (72.34%) | 33 | (75.00%) | 35 | (70.00%) | |
| Chronic pelvic pain history | | | | | | | 0.83 |
| (n = 91) | | | | | | | |
| Yes | 50 | (54.95%) | 22 | (56.41%) | 28 | (53.85%) | |
| No | 41 | (45.05%) | 17 | (43.59%) | 24 | (46.15%) | |
| Endometriosis history | | | | | | | 0.11 |
| (n = 95) | | | | | | | |
| Yes | 28 | (29.47%) | 16 | (39.02%) | 12 | (22.22%) | |
| No | 67 | (70.53%) | 25 | (60.98%) | 42 | (77.78%) | |
| PCOS history (n = 88) | | | | | | | 0.50 |
| Yes | 10 | (11.36%) | 3 | (7.69%) | 7 | (14.29%) | |
| No | 78 | (88.64%) | 36 | (92.31%) | 42 | (85.71%) | |
| Diabetes (n = 108) | | | | | | | 0.15 |
| Yes | 22 | (20.37%) | 6 | (13.04%) | 16 | (25.81%) | |
| No | 86 | (79.63%) | 40 | (86.96%) | 46 | (74.19%) | |
| Hypertension (n = 108) | | | | | | | 0.99 |
| Yes | 25 | (23.15%) | 11 | (23.91%) | 14 | (22.58%) | |
| No | 83 | (76.85%) | 35 | (76.09%) | 48 | (77.42%) | |
| Antibiotics (use within 3 | | | | | | | 0.23 |
| months) (n = 95) | | | | | | | |
| Yes | 23 | (24.21%) | 13 | (30.95%) | 10 | (18.87%) | |
| No | 72 | (75.79%) | 29 | (69.05%) | 43 | (81.13%) | |
| Combined contraceptives | | | | | | | 0.24 |
| Hormonal (n = 81) | | | | | | | |
| Yes | 26 | (32.10%) | 9 | (25.00%) | 17 | (37.78%) | |
| No | 55 | (67.90%) | 27 | (75.00%) | 28 | (62.22%) | |
| Non-hormonal (n = 40) | | | | | | | 0.99 |
| Yes | 1 | (2.50%) | 1 | (5.00%) | 0 | (0.00%) | |
| No | 39 | (97.50%) | 19 | (95.00%) | 20 | (100.00%) | |

Cervicovaginal lavage (CVL) samples were collected by a surgeon during the standard-of-care hysterectomy procedure. Samples were obtained after anesthesia and prior to vaginal sterilization. CVL were collected using a non-lubricated speculum and 10 ml of sterile 0.9% saline solution (Teknova, Hollister, CA) Following collection, samples were immediately placed on ice and frozen at −80° C. within an hour. Prior to analyses, the samples were thawed on ice; centrifuged (700×g for 10 minutes at 4° C.); aliquoted, to prevent multiple freeze-thaw cycles; and stored at −80° C.

Quantification of soluble proteins: The protein concentrations in CVL samples were measured using the Milliplex MAP Magnetic Bead Immunoassays: Human Cytokine Chemokine Panel 1, Human Circulating Cancer Biomarker Panel 1 and Human Immuno-Oncology Checkpoint Protein Panel 1 (Millipore, Billerica, MA) according to the manufacturer's protocol. The levels of 71 proteins (α-fetoprotein (AFP), B- and T-lymphocyte attenuator (BTLA), cancer antigen (CA) 15-3 (CA15-3), CA19-9, CA125, cluster of differentiation (CD) 27 (CD27), CD28, CD40, CD80, CD86, carcinoembryonic antigen (CEA), cytokeratin 19 fragment (CYFRA21-1), epidermal growth factor (EGF), eotaxin/CCL11, Fms-related tyrosine kinase 3 ligand (FIt-3L) basic fibroblast growth factor 2 (FGF-2), fractalkine/CXC3CL1, granulocyte colony-stimulating factor (G-CSF), glucocorticoid-induced TNFR-related protein ligand (GITRL), growth related oncogene α (GROα/CXCL1), granulocyte-macrophage colony-stimulating factor (GM-CSF), human epididymis protein 4 (HE4), hepatocyte growth factor (HGF), herpes virus entry mediator (HVEM), inducible T-cell costimulatory (ICOS), interferon (IFN) α2 (IFNα2), IFNγ, interleukin (IL)-1α (IL-1α), IL-1β, IL-2, IL-4, IL-5, IL-6, IL-7, IL-8/CXCL8, IL-9, IL-10, IL-12 p40, IL-12 p70, IL-13, IL-15, IL-17A, interferon gamma-induced protein 10 (IP-10/CXCL10), lymphocyte activation gene 3 (LAG3), leptin, monocyte chemoattractant protein (MCP)-1 (MCP-1/CCI2), MCP-3/CCL7, macrophage-derived chemokine (MDC/CCL22), macrophage migration inhibitory factor (MIF), macrophage inflammatory protein (MIP)-1α (MIP-1α/CCL3), MIP-1β/CCL4, osteopontin (OPN), programmed cell death protein 1 (PD-1), programmed death ligand 1 (PD-L1), programmed death ligand 2 (PD-L2), platelet-derived growth factor (PDGF) AA (PDGF-AA), PDGF-AB/BB, prolactin, prostate specific antigen (PSA total), regulated on activation normal T-cell expressed and secreted (RANTES/CCL5), Skp1-Cullin-F-box (SCF), soluble protein CD40 ligand (sCD40L), soluble Fas (sFas), soluble Fas ligand (sFasL), transforming growth factor α (TGF-α), T-cell immunoglobulin and mucin domain-containing protein 3 (TIM-3), toll-like receptor 2 (TLR2), tumour necrosis factor (TNF) α (TNFα), TNFβ, TNF-related apoptosis-inducing ligand (TRAIL), vascular endothelial growth factor (VEGF)) were quantified using a Bio-Plex 200 instrument and Bio-Plex Manager 5.0 software (Bio-Rad, Hercules, CA). Levels of IL-36γ (IL-1F9)) were measured in the samples by enzyme-linked immunosorbent assay using Human IL-36γ ELISA kit (RayBiotech, Norcross, GA) in accordance with the manufacturer's instructions. All samples were analyzed in duplicate. The concentrations were determined using a five-parameter logistic regression curve fit. If the concentrations measured were below the detection limit, the value was substituted with 0.5 of the minimum detectable concentration provided in the manufacturer's instructions. The data were normalized using the log₁₀ transformation.

Quantification of soluble metabolites: The soluble metabolites in the CVL samples were determined using a global metabolomics platform at Metabolon, Inc. (Durham, NC). Samples were prepared using the MicroLab STAR® system (Hamilton, Reno, NV). Recovery standards were added for quality control purposes. To recover metabolites and remove protein, the samples were precipitated with methanol under shaking for 2 minutes (Glen Mills GenoGrinder 2000) followed by centrifugation. Samples were then placed on a TurboVap® (Zymark) to remove the organic solvent. The samples were split into five aliquots, one for each of the analyses and one spare. A pooled matrix was generated by mixing a small volume of each sample to serve as a technical replica. Extracted water samples were utilized as process blanks and a mix of quality control standards were selected and added to each sample to monitor instrument performance and aid chromatographic alignment.

All methods utilized a Waters ACQUITY ultra-performance liquid chromatography (UPLC) and a Thermo Scientific Q-exactive high resolution/accurate mass spectrometer interfaced with a heated electrospray ionization (HESI-II) source and Orbitrap mass analyser operated at 35000 mass resolution. The sample extract was dried and then resuspended in solvents compatible with each of the four methods listed below. The resuspension solvents all contained a series of standards at fixed concentrations for chromatographic consistency.

One aliquot was analyzed using acidic positive ion conditions, optimized for hydrophilic compounds. The extract was gradient eluted from a C18 column (Waters UPLC BEH C18-2.1×100 mm, 1.7 μm) using water and methanol, containing 0.05% perfluoropentanoic acid, and 0.1% formic acid. Another aliquot was analyzed using acidic positive ion conditions, optimized for hydrophobic compounds. The extract was gradient eluted from the same C18 column using methanol, acetonitrile, water, 0.05% perfluoropentanoic acid, and 0.01% formic acid and was operated at a higher organic content. The third aliquot was analyzed using basic negative ion optimized conditions using a separated C18 column. The basic extracts were gradient eluted using methanol and water, and 6.5 mM ammonium bicarbonate at pH 8. The fourth aliquot was analyzed via negative ionization following elution from a HILIC column (Water UPLC BEH Amide 2.1×150 mm, 1.7 um) using a gradient consisting of water and acetonitrile with 10 mM ammonium formate, pH 10.8.

Peak analysis and quality control processing were performed by the Metabolon Laboratory Information System for compound identification. The Metabolon's library is able to match compounds to more than 3300 purified standards. In addition, recurrent unknown entities were also reported. Peaks were quantified using area-under-the-curve for relative intensity. The data was normalized by registering the medians of each compound to equal one and normalizing each data point proportionately. The data was transformed using the log₁₀ transformation and autoscaled (mean-centered and divided by the standard deviation of each variable). Percentage fill value data was determined by calculating the percentage of samples that a particular metabolite was detected in for each group (adenomyosis and no adenomyosis).

Partial least squares-discriminant analysis: A partial least squares-discriminant analysis (PLS-DA) was performed using MetaboAnalyst 5.0 to visualize the separation of the two patient groups: adenomyosis (n=46) and no adenomyosis (n=62). PLS-DA is a supervised regression method that aims to plot the greatest separation between groups by finding the maximum covariance between the data and the assigned group.

Volcano plot analysis: An unpaired t-test was performed with a significance value threshold of 0.05 p-value. Fold change analysis compared the absolute value of change between the means of each metabolite/protein between the two groups. The fold-change analysis utilizes the data prior to data transformation and scaling. Data from fold change and t-test analysis was combined to produce volcano plots that depict the significantly up-/down regulated metabolites and immune proteins in adenomyosis compared to no adenomyosis group. The comparison of direction was adenomyosis vs. no adenomyosis and the fold change threshold was 2.0. The analysis was performed using MetaboAnalyst 5.0.

Hierarchical clustering analysis: Partially supervised hierarchical clustering analysis was performed on metabolite and immunoprotein data sets, individually, using MetaboAnalyst 5.0 to produce heatmaps. The metabolites/immune proteins were autoscaled and then Pearson distance measure and Ward linkage was applied to the metabolites/immune proteins. The samples were analyzed both with and without clustering; for those analyzed without clustering, the order of samples remained in the supervised order inputted, which was categorized based on adenomyosis status.

Enrichment analysis: Enrichment analysis was completed in Metaboanalyst 5.0 by comparing the metabolite data to the Small Molecule Pathway Database metabolite set based on normal human metabolic pathways. The enrichment ratio and significance of the enrichment of metabolic pathways were calculated based on the number of metabolites detected within a specific pathway relative to the number of known metabolites in that pathway. The algorithm also considered the relative intensity of the metabolites in adenomyosis compared to no adenomyosis.

Other statistical analyses: Statistical differences between the mean intensities of metabolites among the groups were determined using a t-test. P-values were corrected using the false discovery rate (FDR) method and q-values have been reported. P-values <0.05 were considered statistically significant. Statistical analyses were performed using MetaboAnalyst 5.0 (Pang, 2021). Differences in the demographic, socioeconomic and other patient-related variables between disease groups (adenomyosis vs. no adenomyosis) were tested using Wilcoxon rank sum test for continuous variables and Fisher's exact test for categorical variables.

Study population: In this study, the immunoproteomic and metabolic differences between adenomyosis patients and patients with no adenomyosis was investigated. To do this, clinical cervicovaginal lavage samples were analyzed from 108 women undergoing hysterectomy for benign conditions in the Phoenix area. The women were stratified according to whether they had adenomyosis (n=46) or no adenomyosis (n=62) according to histopathology investigation post-hysterectomy.

Clinical and demographic information for this cohort is reported in Table 1. The mean age of patients involved study was 45.55 years, with no significant difference between groups (p=0.65). All demographic information was found to not be significantly different, such as: race (p=0.75), ethnicity (p=0.52), body mass index (BMI) (p=0.89). Investigation of socioeconomic factors, education level (p=0.20), income (p=0.43), employment status (p=0.65) showed no significant differences. Medical history also revealed no significant differences between the groups: menopausal status (p=0.62), previous dilation and curettage (p=0.80), co-occurring conditions of endometriosis and leiomyoma (uterine fibroids) (p=0.31 and p=0.94, respectively), parity (p=0.09), heavy periods (p=0.61), history of chronic pelvic pain (p=0.83), history of endometriosis (p=0.11), history of polycystic ovary syndrome (PCOS) (p=0.50). Finally, for those patients that had contraceptive data, there was no significant difference found between hormonal (p=0.24) or non-hormonal (p=0.99) contraceptive use between the groups. See Table 2 for more information on hormonal method use within the two patient populations.

TABLE 2
Patient's demographic information, no significance seen between any data,
except hormonal IUD (p = 0.02), but no significant difference seen
overall with hormonal or non-hormonal contraceptive use (p = 0.24
and 0.99, respectively). P-values were calculated using Wilcoxon rank sum
test for continuous variables and Fisher's exact test for categorical variables.

| | All | | Adenomyosis | | No Adenomyosis | | |
|---|---|---|---|---|---|---|---|
| | (n = 108) | | (n = 46) | | (n = 62) | | p-value |
| pH (n = 108) | | | | | | | 0.49 |
| ≤4.5 | 83 | (76.85%) | 37 | (80.43%) | 46 | (74.19%) | |
| >4.5 | 25 | (23.15%) | 9 | (19.57%) | 16 | (26.81%) | |
| Contraceptive use in the | | | | | | | |
| last 6 months | | | | | | | |
| Birth control pill (n = 75) | | | | | | | 0.98 |
| Yes | 12 | (16.00%) | 5 | (14.71%) | 7 | (17.07%) | |
| No | 63 | (84.00%) | 29 | (85.29%) | 34 | (82.93%) | |
| BC/hormone patch (n = 40) | | | | | | | — |
| Yes | 0 | (0.00%) | 0 | (0.00%) | 0 | (0.00%) | |
| No | 40 | (100.00%) | 19 | (100.00%) | 21 | (100.00%) | |
| Depo provera (n = 45) | | | | | | | — |
| Yes | 0 | (0.00%) | 0 | (0.00%) | 0 | (0.00%) | |
| No | 45 | (100.00%) | 21 | (100.00%) | 24 | (100.00%) | |
| Hormone receptor (n = 39) | | | | | | | 0.50 |
| Yes | 2 | (5.13%) | 2 | (10.00%) | 0 | (0.00%) | |
| No | 37 | (94.87%) | 18 | (90.00%) | 19 | (100.00%) | |
| Hormone tx (n = 49) | | | | | | | 0.73 |
| Yes | 10 | (20.41%) | 4 | (17.39%) | 6 | (23.08%) | |
| No | 39 | (79.59%) | 19 | (82.61%) | 20 | (76.92%) | |
| Implant last (n = 37) | | | | | | | — |
| Yes | 0 | (0.00%) | 0 | (0.00%) | 0 | (0.00%) | |
| No | 37 | (100.00%) | 17 | (100.00%) | 20 | (100.00%) | |
| Paragard (n = 40) | | | | | | | 0.99 |
| Yes | 1 | (2.50%) | 1 | (5.00%) | 0 | (0.00%) | |
| No | 39 | (97.50%) | 19 | (95.00%) | 20 | (100.00%) | |
| Hormone IUD (n = 42) | | | | | | | 0.02 |
| Yes | 9 | (21.43%) | 1 | (5.00%) | 8 | (36.36%) | |
| No | 33 | (78.57%) | 19 | (95.00%) | 14 | (63.64%) | |
| Vaginal ring (n = 38) | | | | | | | — |
| Yes | 0 | (0.00%) | 0 | (0.00%) | 0 | (0.00%) | |
| No | 38 | (100.00%) | 19 | (100.00%) | 19 | (100.00%) | |
| Surgical contraception | | | | | | | 0.24 |
| (n = 61) | | | | | | | |
| Tubes Tied | 25 | (40.98%) | 11 | (44.00%) | 14 | (38.89%) | |
| Essure | 5 | (8.20%) | 3 | (12.00%) | 2 | (5.56%) | |
| Both ovaries removed | 1 | (1.64%) | 0 | (0.00%) | 1 | (2.78%) | |
| Tube and ovaries removed | 2 | (3.28%) | 2 | (8.00%) | 0 | (0.00%) | |
| None | 28 | (45.90%) | 9 | (36.00%) | 19 | (52.78%) | |
| Uterine manipulator used | | | | | | | 0.95 |
| (n = 104) | | | | | | | |
| Fornisee | 61 | (58.65%) | 28 | (63.63%) | 33 | (55.00%) | |
| Sacrocervicopexy | 0 | (0.00%) | 0 | (0.00%) | 0 | (0.00%) | |
| Delineator | 31 | (29.81%) | 12 | (27.27%) | 19 | (31.67%) | |
| V-care | 5 | (4.81%) | 2 | (4.55%) | 3 | (5.00%) | |
| RUMI | 6 | (5.77%) | 2 | (4.55%) | 4 | (6.67%) | |
| Sponge stick with 4 × 4 | 1 | (0.96%) | 0 | (0.00%) | 1 | (1.67%) | |
| sponge | | | | | | | |

Immune protein profiling of cervicovaginal samples: To study the immunoproteomic differences between with and without adenomyosis, the levels of 72 soluble proteins were investigated in the cervicovaginal lavage (CVL) samples, including cytokines, chemokines, growth factors, circulating cancer biomarkers, and immune checkpoint proteins. Hierarchical clustering analysis was not able to correctly predict adenomyosis based on the global immunoproteomic profiles.

To identify proteins in the CVL samples that were altered in women diagnosed with adenomyosis unpaired T-tests and fold change analysis were performed. The analysis revealed that eight soluble proteins were significantly (p<0.05) different in the adenomyosis group compared to no adenomyosis group (FIG. 1A). Cell surface antigen CA19-9 (p=0.031) and chemokines GRO (p=0.021) and IP-10 (p=0.015) were all significantly downregulated in adenomyosis compared to no adenomyosis (FIG. 1B). The significantly upregulated proteins in adenomyosis included cell surface antigen CEA (p=0.042) and the cytokines: IL-9 (p=0.047), IL-13 (p=0.042), IL-36γ (p=0.017), and TNFβ (p=0.038) (FIG. 1C). These cytokines, specifically IL-9 and IL-13, play an important role in type 2 immunity and act as chemoattractants for several immune cells, including mast cells. In contrast, pro-inflammatory cytokines such as IL-1α, IL-1γ, IL-6, IL-8, MIP-1β, RANTES, and TNFα were not significantly increased. The levels of CA125 and HE4 were not significantly different between adenomyosis and no adenomyosis groups (p=0.117 and p=0.09, respectively) (see Table 3). Overall, only a few proteins were altered in the CVL samples, mostly immunoregulatory and involved in type II immune responses.

TABLE 3
Detected levels of soluble immune proteins tested in adenomyosis
and no adenomyosis. P-value calculated by unpaired t-tests.
Mean levels determined from relative abundance.

| | | Mean of | |
|---|---|---|---|
| Protein | p-value | Adenomyosis | Mean of No Adenomyosis |
| IL36γ | 0.045553 | 559.4 | 147 |
| EGF | 0.198112 | 34.62 | 22.31 |
| FGF-2 | 0.611828 | 49.36 | 79.97 |
| Eotaxin | 0.279612 | 14.92 | 10.59 |
| TGF-alpha | 0.079516 | 6.544 | 9.657 |
| G-CSF | 0.89471 | 610.4 | 591.8 |
| Flt-3L | 0.644087 | 12.04 | 11.03 |
| GM-CSF | 0.359018 | 5.153 | 7.093 |
| Fractalkine | 0.095601 | 79.83 | 53.94 |
| IFNalpha2 | 0.538759 | 7.441 | 5.983 |
| IFNgamma | 0.451234 | 1.619 | 1.288 |
| GRO | 0.032738 | 1755 | 2907 |
| IL-10 | 0.285289 | 21.94 | 9.862 |
| MCP-3 | 0.909557 | 13.38 | 13.91 |
| IL-12p40 | 0.325795 | 5.576 | 4.929 |
| MDC | 0.248852 | 81.14 | 101.4 |
| IL-12p70 | 0.511359 | 2.689 | 2.099 |
| PDGF-AA | 0.150148 | 55.15 | 29.44 |
| IL-13 | 0.037757 | 3.736 | 1.281 |
| PDGF-AB/BB | 0.069056 | 171.5 | 107.1 |
| IL-15 | 0.132454 | 3.053 | 1.499 |
| sCD40L | 0.117259 | 38.91 | 14.12 |
| IL-17A | 0.576373 | 2.19 | 1.8 |
| IL-1alpha | 0.034741 | 313.4 | 135.4 |
| IL-9 | 0.055267 | 1.992 | 0.887 |
| IL-1beta | 0.924819 | 128.3 | 135.5 |
| IL-2 | 0.450468 | 0.8741 | 1.145 |
| IL-4 | 0.056788 | 112.8 | 26.73 |
| IL-5 | 0.23494 | 1.049 | 0.667 |
| IL-6 | 0.187843 | 47.41 | 26.47 |
| IL-7 | 0.170099 | 3.898 | 2.874 |
| IL-8 | 0.500485 | 1379 | 1577 |
| IP-10 | 0.206596 | 307.5 | 543.2 |
| MCP-1 | 0.162198 | 287.6 | 453.5 |
| MIP-1alpha | 0.381464 | 17.1 | 42.19 |
| MIP-1beta | 0.714605 | 53.32 | 41.85 |
| RANTES | 0.253631 | 280.6 | 163.5 |
| TNFalpha | 0.434426 | 9.337 | 19.21 |
| TNFβ | 0.0435 | 18.64 | 1.777 |
| AFP | 0.594755 | 65.14 | 53.55 |
| PSA (total) | 0.43031 | 1094 | 746.7 |
| CA15-3 | 0.647676 | 0.6099 | 0.6774 |
| CA19-9 | 0.821307 | 33422 | 38318 |
| MIF | 0.101964 | 878.6 | 461.6 |
| TRAIL | 0.991201 | 29.32 | 29.44 |
| Leptin | 0.105963 | 401.2 | 201.6 |
| sFasL | 0.262324 | 10.66 | 4.353 |
| CEA | 0.031802 | 46924 | 18802 |
| CA125 | 0.117251 | 2332 | 972.2 |
| HGF | 0.998049 | 286.2 | 286.4 |
| sFas | 0.515423 | 135.9 | 114.9 |
| Prolactin | 0.567443 | 486.6 | 339.7 |
| SCF | 0.962434 | 5.072 | 4.967 |
| CYFRA 21-1 | 0.707088 | 502904 | 610649 |
| OPN | 0.290598 | 303.5 | 439.9 |
| HE4 | 0.090006 | 63302 | 86122 |
| VEGF | 0.648692 | 60.38 | 76.44 |
| BTLA | 0.58668 | 33.21 | 59.19 |
| CD27 | 0.736561 | 10.98 | 10.34 |
| CD28 | 0.289159 | 513.1 | 655.2 |
| TIM-3 | 0.927166 | 26.22 | 26.81 |
| HVEM | 0.299733 | 833.8 | 724.4 |
| CD40 | 0.341138 | 169.1 | 142.9 |
| LAG-3 | 0.658119 | 372.5 | 557.3 |
| TLR-2 | 0.104738 | 145 | 199.6 |
| GITRL | 0.129388 | 20.83 | 12.29 |
| PD-1 | 0.834723 | 11.42 | 10.57 |
| CD80 | 0.423263 | 6.495 | 10.47 |
| CD86 | 0.694814 | 36.24 | 33.06 |
| PD-L1 | 0.401405 | 2.29 | 1.319 |
| PD-L2 | 0.953805 | 86.82 | 85.19 |
| ICOS | 0.232938 | 112.8 | 30.39 |

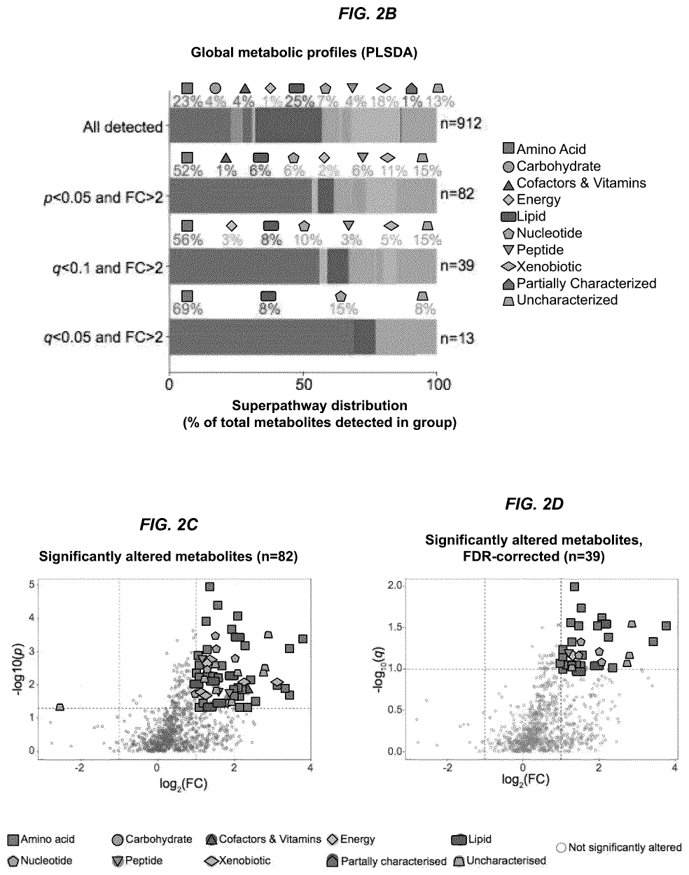

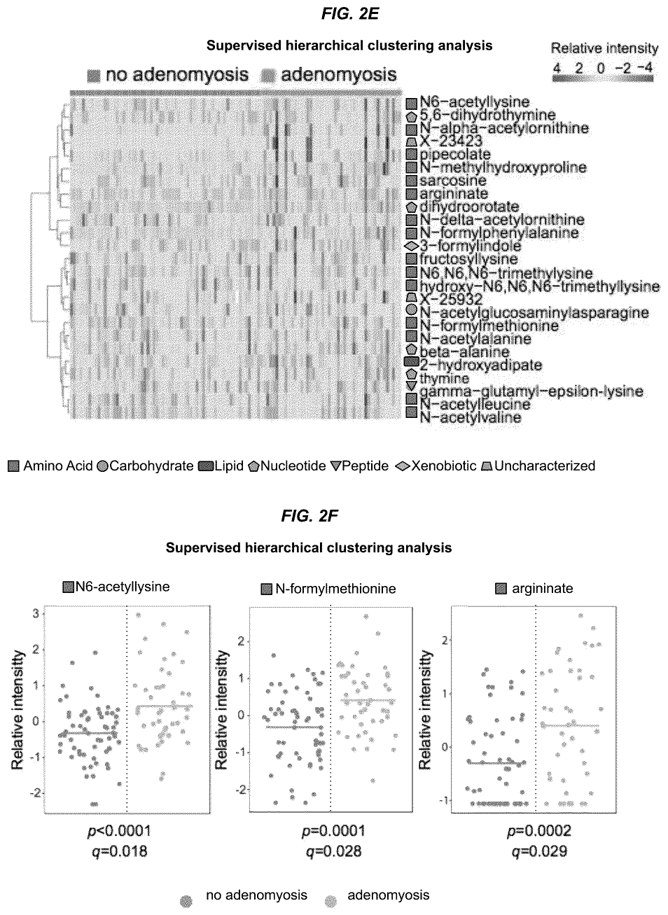

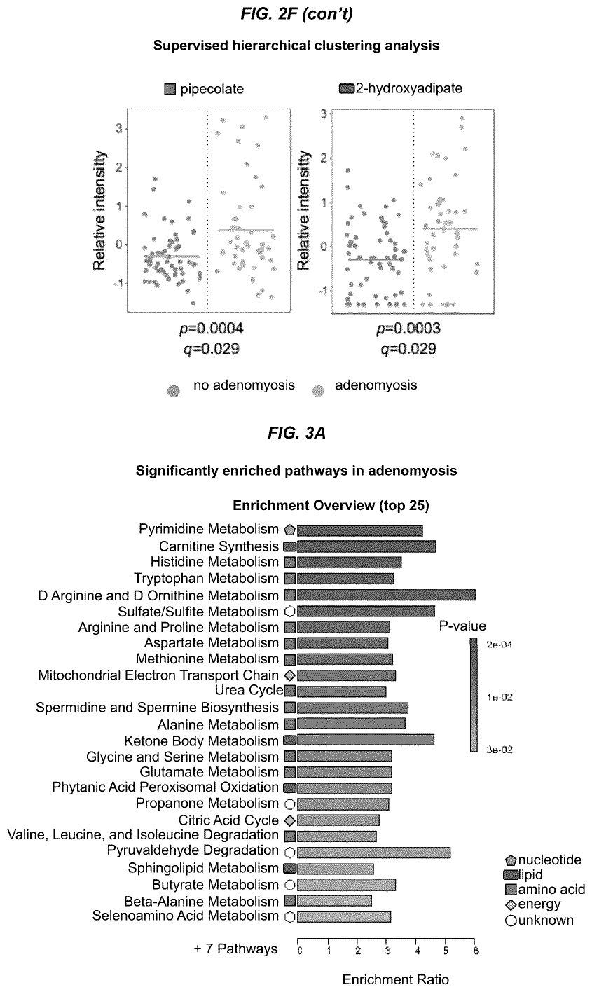

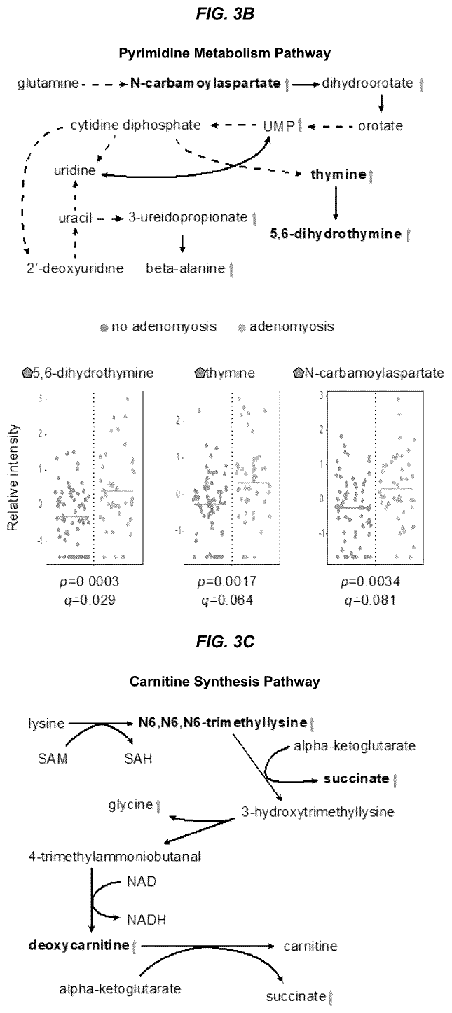

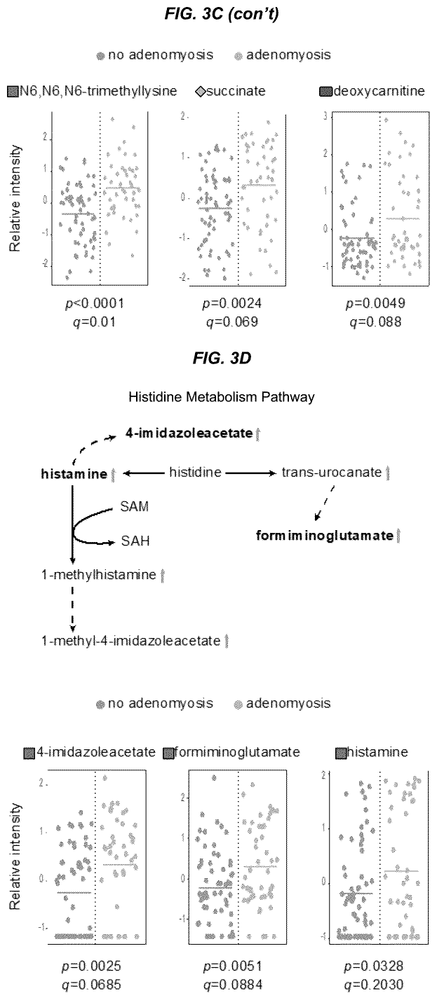

Global metabolomic profiling of cervicovaginal samples: Next, liquid chromatography and mass spectroscopy were used to determine the metabolic profiles of the cervicovaginal lavages collected from women with benign conditions and to identify metabolic differences that may be present between women with and without adenomyosis. The global metabolic analysis identified 912 metabolites, 784 fully characterized compounds, and 128 partially characterizable or uncharacterized within the cervicovaginal lavage samples (FIG. 3A-3D). Global metabolic profiles of patients with adenomyosis and no adenomyosis were created by the data reduction method partial least square-discriminant analysis (PLS-DA); comparison of these profiles revealed separation between the two patient groups. To construct the PLS-DA, two components were used, component 1 and component 2, accounting for 11.6% and 11.1% of the data, respectively. slight separation with some overlap in overall metabolic profiles was observed between the adenomyosis and no adenomyosis groups (FIG. 2A).

The 912 detected metabolites included amino acids (n=206, 23%), carbohydrates (n=34, 4%), cofactors and vitamins (n=32, 4%), energy (n=12, 1%), lipids (n=228, 25%), nucleotides (n=67, 7%), peptides (n=40, 4%), xenobiotics (n=165, 18%), partially characterized metabolites (n=9, 1%) and uncharacterized metabolites (n=119, 13%). Analysis of superpathway distribution among the metabolites detected revealed a distribution across all superpathways-with amino acids accounting for only 23% of all metabolites. Next, unpaired T-tests were performed to determine which metabolites were significant between adenomyosis and no adenomyosis, as well as fold change analysis to identify metabolites that were up/downregulated. The threshold for fold-change analysis was set at 2.0 or greater to be classed as ‘altered’.

A large proportion of significantly (p<0.05) altered metabolites were amino acids (n=43) accounting for more than half of those metabolites (52%) that were altered in adenomyosis compared to no adenomyosis. Furthermore, a false-discovery rate (FDR) correction of 10% and 5% was performed; this amino acid signature remained dominant, with amino acids accounting for 56% (n=22) and 69% (n=9), respectively (FIG. 2B). Fold change analysis and T-tests combined to produce volcano plots revealed that 82 metabolites were significantly altered (p<0.05 and fold change>2) in adenomyosis patients compared to no adenomyosis, with 1 metabolite being downregulated and 81 upregulated (FIG. 2C, Table 4). Statistical analysis with FDR-correction of 10% resulted in 39 significantly upregulated metabolites in adenomyosis (FIG. 2D).

Table 4 shows significantly altered metabolites (p<0.05, FC>2.0) in adenomyosis compared to no adenomyosis. Unpaired t-tests for significance levels, fold change analysis to determine up/downregulation in adenomyosis compared to no adenomyosis, FDR-correction to produce q-values. % fill values to determine the amount of adenomyosis samples and no adenomyosis samples each metabolite was detected in.

| | | | | | % fill values | |
|---|---|---|---|---|---|---|
| | Fold | p- | q- | Super | | No |
| Metabolite name | Change | value | value | pathway | Adenomyosis | Adenomyosis |
| N6,N6,N6- | 2.56 | <0.0001 | 0.010 | Amino | 100 | 100 |
| trimethyllysine | | | | acid | | |
| N-methylhydroxyproline | 4.17 | <0.0001 | 0.025 | Amino | 52 | 23 |
| | | | | acid | | |
| N6-acetyllysine | 2.91 | <0.0001 | 0.018 | Amino | 100 | 97 |
| | | | | acid | | |
| N-formylmethionine | 2.41 | 0.0001 | 0.028 | Amino | 100 | 97 |
| | | | | acid | | |
| argininate | 3.83 | 0.0002 | 0.029 | Amino | 85 | 60 |
| | | | | acid | | |
| X-23423 | 7.23 | 0.0003 | 0.029 | Unknown | 37 | 19 |
| 2-hydroxyadipate | 3.99 | 0.0003 | 0.029 | Lipid | 85 | 81 |
| 5,6-dihydrothymine | 2.79 | 0.0003 | 0.029 | Nucleotide | 91 | 77 |
| pipecolate | 13.64 | 0.0004 | 0.029 | Amino | 100 | 100 |
| | | | | acid | | |
| sarcosine | 4.78 | 0.0006 | 0.041 | Amino | 87 | 79 |
| | | | | acid | | |
| dihydroorotate | 2.88 | 0.0008 | 0.046 | Nucleotide | 70 | 35 |
| N-alpha-acetylornithine | 10.75 | 0.0008 | 0.046 | Amino | 48 | 24 |
| | | | | acid | | |
| N-formylphenylalanine | 2.45 | 0.0009 | 0.046 | Amino | 67 | 39 |
| | | | | acid | | |
| N-acetylleucine | 2.10 | 0.0013 | 0.057 | Amino | 96 | 85 |
| | | | | acid | | |
| thymine | 4.03 | 0.0017 | 0.064 | Nucleotide | 93 | 87 |
| gamma-glutamyl- | 2.28 | 0.0017 | 0.064 | Peptide | 93 | 90 |
| epsilon-lysine | | | | | | |
| 3-formylindole | 2.54 | 0.0018 | 0.068 | Xenobiotic | 85 | 73 |
| succinate | 2.47 | 0.0024 | 0.069 | Energy | 100 | 100 |
| X-23908 | 2.78 | 0.0024 | 0.069 | Unknown | 24 | 6 |
| 4-imidazoleacetate | 2.14 | 0.0025 | 0.069 | Amino | 74 | 58 |
| | | | | acid | | |
| N-methylproline | 2.95 | 0.0025 | 0.069 | Amino | 96 | 74 |
| | | | | acid | | |
| X-25958 | 6.86 | 0.0028 | 0.070 | Unknown | 30 | 10 |
| N-carbamoylaspartate | 2.48 | 0.0034 | 0.081 | Nucleotide | 98 | 85 |
| isobutyrylglycine (C4) | 2.03 | 0.0043 | 0.085 | Amino | 61 | 34 |
| | | | | acid | | |
| X-25047 | 4.20 | 0.0044 | 0.085 | Unknown | 87 | 79 |
| X-24246 | 6.63 | 0.0045 | 0.085 | Unknown | 50 | 34 |
| N6,N6-dimethyllysine | 2.42 | 0.0046 | 0.087 | Amino | 74 | 56 |
| | | | | acid | | |
| deoxycarnitine | 3.96 | 0.0049 | 0.088 | Lipid | 100 | 100 |
| 2-oxoarginine | 2.63 | 0.0050 | 0.088 | Amino | 100 | 100 |
| | | | | acid | | |
| N-acetyl-2- | 2.74 | 0.0051 | 0.088 | Amino | 87 | 73 |
| aminoadipate | | | | acid | | |
| formiminoglutamate | 2.11 | 0.0051 | 0.088 | Amino | 91 | 84 |
| | | | | acid | | |
| 2-hydroxy-4- | 2.91 | 0.0054 | 0.089 | Amino | 89 | 82 |
| (methylthio)butanoic | | | | acid | | |
| acid | | | | | | |
| N-acetylmethionine | 2.08 | 0.0060 | 0.096 | Amino | 100 | 100 |
| | | | | acid | | |
| 2-hydroxyglutarate | 2.53 | 0.0064 | 0.098 | Lipid | 100 | 100 |
| N-acetylglutamate | 2.11 | 0.0065 | 0.098 | Amino | 100 | 100 |
| | | | | acid | | |
| X-24724 | 2.38 | 0.0069 | 0.098 | Unknown | 78 | 68 |
| methyl-4- | 3.09 | 0.0070 | 0.098 | Xenobiotic | 46 | 21 |
| hydroxybenzoate | | | | | | |
| sulfate | | | | | | |
| N-carbamoylsarcosine | 2.06 | 0.0072 | 0.098 | Amino | 52 | 23 |
| | | | | acid | | |
| 4- | 5.13 | 0.0073 | 0.098 | Amino | 74 | 55 |
| hydroxyphenylacetate | | | | acid | | |
| 3-(4-hydroxyphenyl) | 8.66 | 0.0080 | 0.101 | Xenobiotic | 46 | 24 |
| propionate | | | | | | |
| N-acetyltaurine | 2.06 | 0.0084 | 0.104 | Amino | 100 | 100 |
| | | | | acid | | |
| 2-isopropylmalate | 4.80 | 0.0085 | 0.104 | Xenobiotic | 61 | 39 |
| 1-methylhistamine | 8.80 | 0.0089 | 0.107 | Amino | 57 | 40 |
| | | | | acid | | |
| alpha- | 2.85 | 0.0097 | 0.113 | Amino | 100 | 98 |
| hydroxyisocaproate | | | | acid | | |
| citraconate/glutaconate | 2.76 | 0.0098 | 0.113 | Energy | 100 | 100 |
| Fibrinopeptide A | 2.89 | 0.0107 | 0.122 | Peptide | 11 | 2 |
| phenethylamine | 4.21 | 0.0113 | 0.122 | Amino | 83 | 77 |
| | | | | acid | | |
| alpha- | 4.50 | 0.0123 | 0.128 | Amino | 100 | 98 |
| hydroxyisovalerate | | | | acid | | |
| N-acetyl-cadaverine | 9.80 | 0.0124 | 0.128 | Amino | 87 | 68 |
| | | | | acid | | |
| N2-acetyl,N6,N6- | 3.93 | 0.0135 | 0.135 | Amino | 70 | 52 |
| dimethyllysine | | | | acid | | |
| X-23738 | 2.87 | 0.0137 | 0.135 | Unknown | 72 | 56 |
| serotonin | 2.12 | 0.0142 | 0.136 | Amino | 11 | 0 |
| | | | | acid | | |
| pantoate | 4.86 | 0.0143 | 0.136 | Cofactors | 67 | 61 |
| | | | | & Vitamins | | |
| X-19913 | 2.17 | 0.0145 | 0.136 | Unknown | 80 | 71 |
| 2-piperidinone | 3.98 | 0.0145 | 0.136 | Xenobiotic | 100 | 100 |
| N,N-dimethyl-pro-pro | 3.03 | 0.0145 | 0.136 | Peptide | 83 | 71 |
| 3-phenylpropionate | 5.06 | 0.0161 | 0.145 | Xenobiotic | 24 | 10 |
| (hydrocinnamate) | | | | | | |
| diacetylspermidine | 2.25 | 0.0170 | 0.148 | Amino | 63 | 48 |
| | | | | acid | | |
| Fibrinopeptide A | 3.64 | 0.0176 | 0.150 | Peptide | 15 | 8 |
| (3-16)** | | | | | | |
| N-acetylhistamine | 10.55 | 0.0194 | 0.158 | Amino | 54 | 47 |
| | | | | acid | | |
| X-21796 | 2.46 | 0.0198 | 0.159 | Unknown | 89 | 71 |
| orotidine | 2.03 | 0.0203 | 0.159 | Nucleotide | 98 | 94 |
| tyramine | 4.22 | 0.0206 | 0.159 | Amino | 87 | 81 |
| | | | | acid | | |
| tryptamine | 3.86 | 0.0206 | 0.159 | Amino | 37 | 16 |
| | | | | acid | | |
| X-25810 | 2.28 | 0.0217 | 0.162 | Unknown | 78 | 66 |
| 4-acetamidophenol | 3.52 | 0.0241 | 0.172 | Xenobiotic | 63 | 44 |
| 5-aminovalerate | 5.79 | 0.0308 | 0.204 | Amino | 59 | 45 |
| | | | | acid | | |
| N-acetylaspartate | 3.54 | 0.0314 | 0.204 | Amino | 100 | 100 |
| (NAA) | | | | acid | | |
| Fibrinopeptide A, | 3.37 | 0.0314 | 0.204 | Peptide | 17 | 3 |
| desala(1) | | | | | | |
| imidazole propionate | 3.66 | 0.0321 | 0.207 | Amino | 96 | 98 |
| | | | | acid | | |
| histamine | 3.44 | 0.0328 | 0.210 | Amino | 76 | 66 |
| | | | | acid | | |
| 1-methyl-5- | 2.24 | 0.0339 | 0.212 | Amino | 100 | 94 |
| imidazolelactate | | | | acid | | |
| X-24410 | 3.75 | 0.0343 | 0.212 | Unknown | 43 | 34 |
| stachydrine | 2.65 | 0.0377 | 0.218 | Xenobiotic | 100 | 100 |
| 2-keto-3-deoxy- | 3.45 | 0.0378 | 0.218 | Xenobiotic | 63 | 50 |
| gluconate | | | | | | |
| margaroylcarnitine | 2.95 | 0.0382 | 0.218 | Lipid | 46 | 27 |
| (C17) | | | | | | |
| 1-palmitoyl-2-linoleoyl- | 2.54 | 0.0424 | 0.236 | Lipid | 76 | 65 |
| GPE (16:0/18:2) | | | | | | |
| dopamine 3-O-sulfate | 2.12 | 0.0438 | 0.239 | Amino | 80 | 63 |
| | | | | acid | | |
| indolepropionate | 2.52 | 0.0440 | 0.239 | Amino | 22 | 15 |
| | | | | acid | | |
| N-acetylputrescine | 4.34 | 0.0444 | 0.239 | Amino | 100 | 100 |
| | | | | acid | | |
| X-21803 | 0.17 | 0.0445 | 0.239 | Unknown | 17 | 27 |
| 2-hydroxy-3- | 5.07 | 0.0475 | 0.247 | Amino | 83 | 79 |
| methylvalerate | | | | acid | | |

Supervised hierarchical clustering analysis (HCA) was performed to depict the levels of metabolites within individual samples and identify whether a clear clustering pattern emerged between adenomyosis and no adenomyosis. HCA of the top 25 significant metabolites produced a heat map with a distinct signature, that reflected an enriched metabolic pattern, for adenomyosis compared to no adenomyosis (FIG. 2E).

N6-acetyllysine (p<0.0001, q=0.018), N-formylmethionine (p=0.0001 and q=0.028), argininate (p=0.0002 and q=0.029), and pipecolate (p=0.0004 and q=0.029) were some of the significantly upregulated amino acids in adenomyosis that contributes to the amino acid signatures. These amino acids were selected as they had the lowest p-values, were still significant after FDR-correction of 5% and were detected in at least 85% of adenomyosis patients (FIG. 2F). Within the lipid superpathway, 2-hydroxyadipate (p=0.0003 and q=0.029) was the only lipid that was significantly upregulated, remained significant after FDR-correction to 5%, and was detected in at least 85% of adenomyosis patients (FIG. 2F). To summarize, the analyses show that adenomyosis is distinguished by a unique metabolic signature compared to patients with other benign conditions, with this signature appearing to be created by significantly upregulated amino acids.

Metabolite enrichment analysis: Next, an enrichment analysis was performed to identify the metabolic pathways that were likely to be altered in adenomyosis compared to no adenomyosis based on the relative level of metabolites within each group. This analysis revealed that 32 metabolic pathways were significantly (p<0.05) enriched in adenomyosis compared to no adenomyosis (FIG. 3A). The figure shows the top 25 enriched pathways that were associated with nucleotide (n=1), lipid (n=6), amino acid (n=17), or energy metabolism (n=3), and some were not characterizable to a particular superpathway (n=5). The nucleotide pathway pyrimidine metabolism (p<0.0001), the lipid pathway carnitine synthesis (p<0.0001), and the amino acid pathways histidine metabolism (p<0.0001), and tryptophan metabolism (p<0.0001) were among the most significantly enriched pathways (FIGS. 3A, 3B, 3C, and 3D). Scatter plots are shown that represent key metabolites from the pathways, these were the most significant (p<0.05), remained significant after FDR-correction to 10% (q<0.1), and were detected in at least 85% of adenomyosis patients. 5,6-dihydrothymine (p=0.0003 and q=0.029), thymine (p=0.0017 and q=0.064), and N-carbamoylaspartate (p=0.0034 and q=0.081) were all key nucleotides belonging to the pyrimidine metabolism pathway that were the most significant (p<0.05), remained significant after FDR-correction to 10% (q<0.1), and were detected in at least 85% of adenomyosis patients (FIG. 3B). For carnitine synthesis, key metabolites were N6,N6,N6-trimethyllysine (p<0.0001 and q=0.01), succinate (p=0.0024 and q=0.069), and deoxycarnitine (p=0.0049 and q=0.088) (FIG. 3C). From histidine metabolism, 4-imidazoleacetate (p=0.0025 and q=0.0685), formiminoglutamate (p=0.0051 and q=0.0884), and histamine (p=0.0328 and q=0.2030) were key metabolites, they remained significant after FDR-correction to 10% (q<0.1), except histamine, and were detected in at least 70% of adenomyosis patients (FIG. 3D). In total, enrichment analysis revealed that a number of different pathways are enriched within the adenomyosis population, with the majority of these belonging to the amino acid superpathway, and pyrimidine metabolism and carnitine synthesis are the most significantly enriched pathways.

### EXAMPLE 2

The following is a non-limiting example of the present invention. It is to be understood that said example is not intended to limit the present invention in any way. Equivalents or substitutes are within the scope of the present invention.

During a yearly check-up, a patient reports symptoms of heavy menstrual bleeding, severe cramping, and pain during intercourse.The doctor conducts a routine pelvic exam and ultrasound. To obtain a more definitive diagnosis, the doctor suggests collecting a cervicovaginal lavage (CVL) sample for biomarker testing, which the patient agrees to. The sample is sent to a lab where altered biomarkers associated with adenomyosis are detected after a week, confirming the diagnosis of adenomyosis.

The patient schedules another appointment with her doctor to discuss treatment options. The doctor explains that there are various options available depending on the severity of symptoms and the patient's preferences. To start, the doctor recommends using pain medication and hormonal therapy. However, the doctor does note that surgical interventions like endometrial ablation, myomectomy, or hysterectomy may need to be considered if the patient's symptoms do not subside. The patient decides to start a hormonal contraceptive and, after six months of treatment, experiences symptom relief.

### Embodiments

The following embodiments are intended to be illustrative only and not to be limiting in any way.

Embodiment 1: A non-invasive method of diagnosing a benign gynecologic condition in a patient, the method comprising: a) determining the patient's levels of five or more biomarkers comprising protein biomarkers, metabolites biomarkers, or a combination thereof by: i) obtaining a biological sample from the patient; and ii) measuring the levels of five or more biomarkers in the sample obtained in (i); and b) diagnosing the patient with the benign gynecologic conditions if the patient has levels of at least five or more biomarkers are altered from a predetermined threshold.

Embodiment 2: The method of embodiment 1, wherein the benign gynecologic conditions is endometriosis, adenomyosis, fibroids, or a combination thereof

Embodiment 3: The method of embodiment 1 or embodiment 2, wherein the biological sample comprises a cervicovaginal lavage (CVL) sample, a urine sample, a vaginal swab, or a cervicovaginal secretion; wherein the cervicovaginal secretion is collected via a self collected lavage or a menstrual cup.

Embodiment 4: A non-invasive method of diagnosing adenomyosis in a patient, the method comprising: a) determining the patient's levels of five or more biomarkers comprising protein biomarkers, metabolites biomarkers, or a combination thereof by: i) obtaining a biological sample from the patient; and ii) measuring the levels of five or more biomarkers in the sample obtained in (i); and b) diagnosing the patient with adenomyosis if the patient has levels of at least five or more biomarkers are altered from a predetermined threshold.

Embodiment 5: The method of embodiment 4, wherein the biological sample comprises a cervicovaginal lavage (CVL) sample, a urine sample, a vaginal swab or vaginal fluid, or a cervicovaginal secretion; wherein the cervicovaginal secretion is collected via a self collected lavage or a menstrual cup.

Embodiment 6: A non-invasive method of diagnosing adenomyosis in a patient, the method comprising: a) determining the patient's levels of five or more biomarkers comprising protein biomarkers, metabolites biomarkers, or a combination thereof by: i) obtaining a cervicovaginal lavage (CVL) sample from the patient; and ii) measuring the levels of five or more biomarkers in the sample obtained in (i); and b) diagnosing the patient with adenomyosis if the patient has levels of at least five or more biomarkers are altered from a predetermined threshold.

Embodiment 7: The method of any one of embodiments 1-6, wherein the method comprises measuring the levels of ten or more biomarkers in the sample obtained in (i).

Embodiment 8: The method of any one of embodiments 1-6, wherein the method comprises measuring the levels of fifteen or more biomarkers in the sample obtained in (i).

Embodiment 9: The method of any one of embodiments 1-6, wherein the method comprises measuring the levels of twenty or more biomarkers in the sample obtained in (i).

Embodiment 10: The method of any one of embodiments 1-9, wherein the patient is diagnosed with adenomyosis if the levels of at least ten or more biomarkers are altered from a predetermined threshold.

Embodiment 11: The method of any one of embodiments 1-9, wherein the patient is diagnosed with adenomyosis if the levels of at least fifteen or more biomarkers are altered from a predetermined threshold.

Embodiment 12: The method of any one of embodiments 1-9, wherein the patient is diagnosed with adenomyosis if the levels of at least twenty or more biomarkers are altered from a predetermined threshold.

Embodiment 13: The method of any one of embodiments 1-12, wherein the protein biomarkers comprise cell surface antigen biomarkers, chemokine biomarkers, or cytokine biomarkers.

Embodiment 14: The method of embodiment 13, wherein the cell surface antigen biomarkers comprise carbohydrate antigen 19-9 (CA19-9), carcinoembryonic antigen (CEA), or a combination thereof.

Embodiment 15: The method of embodiment 14, wherein the cell surface antigen biomarker CA19-9 is downregulated in patients diagnosed with adenomyosis compared to patients without adenomyosis.

Embodiment 16: The method of embodiment 14, wherein the cell surface antigen biomarker CEA is upregulated in patients diagnosed with adenomyosis compared to patients without adenomyosis.

Embodiment 17: The method of embodiment 16, wherein the chemokine biomarkers comprise growth regulated oncogene (GRO), interferon γ-induced protein 10 kDa (IP-10), or a combination thereof.

Embodiment 18: The method embodiment 14 or embodiment 17, wherein the chemokine biomarkers are downregulated in patients diagnosed with adenomyosis compared to patients without adenomyosis.

Embodiment 19: The method of embodiment 13, wherein the cytokine biomarkers comprise Interleukin (IL)-9, IL-13, IL-36γ, tumor necrosis factor-beta (TNFβ), or a combination thereof.

Embodiment 20: The method of embodiment 13 or embodiment 19, wherein the cytokine biomarkers are upregulated in patients diagnosed with adenomyosis compared to patients without adenomyosis.

Embodiment 21: The method of any one of embodiments 1-20, wherein the metabolite biomarkers comprise amino acid biomarkers, lipid biomarkers, or a combination thereof.

Embodiment 22: The method of embodiment 21, wherein the amino acid biomarkers comprise N6-acetyllysine, N-formylmethionine, argininate, pipecolate, or a combination thereof.

Embodiment 23: The method of embodiment 21 or embodiment 22, wherein the amino acid biomarkers are upregulated in patients diagnosed with adenomyosis compared to patients without adenomyosis.

Embodiment 24: The method of embodiment 21, wherein the lipid biomarker comprises 2-hydroxyadipic.

Embodiment 25: The method of embodiment 21 or embodiment 24, wherein the lipid biomarker is upregulated in patients diagnosed with adenomyosis compared to patients without adenomyosis.

Embodiment 26: A method comprising: a) obtaining a biological sample from a patient; b) producing a profile of the biological sample collected in (a) by detecting at least five or more biomarkers comprising protein biomarkers, metabolite biomarkers, or a combination thereof; and c) analyzing the biological sample profile produced in (b).

Embodiment 27: The method of embodiment 26, wherein the biological sample comprises a cervicovaginal lavage (CVL) sample, a urine sample, a vaginal swab or vaginal fluid, or cervicovaginal secretion; wherein the cervicovaginal secretion is collected via a self collected lavage or a menstrual cup.

Embodiment 28: A method comprising: a) obtaining a cervicovaginal lavage (CVL) sample from a patient; b) producing a profile of the CVL sample collected in (a) by detecting at least five or more biomarkers comprising protein biomarkers, metabolite biomarkers, or a combination thereof; and c) analyzing the CVL sample profile produced in (b).

Embodiment 29: The method of any one of embodiments 26-28, wherein producing a profile comprises detecting at least ten or more biomarkers.

Embodiment 30: The method of any one of embodiments 26-28, wherein producing a profile comprises detecting at least fifteen or more biomarkers.

Embodiment 31: The method of any one of embodiments 26-28, wherein producing a profile comprises detecting at least twenty or more biomarkers.

Embodiment 32: The method of any one of embodiments 26-31, wherein the biomarkers comprise N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, phenethylamine, or a combination thereof.

Embodiment 33: A method of treating a benign gynecologic condition in a patient in need thereof, the method comprising: a) diagnosing the benign gynecologic condition in the patient by: i) obtaining a biological sample from the patient; ii) measuring the levels of five or more biomarkers in the sample obtained in (i); and iii) diagnosing the patient with the benign gynecologic condition if the patient has levels of at least five or more biomarkers altered from a predetermined threshold; and b) administering a therapeutic amount of a treatment to the patient if the patient is diagnosed with a benign gynecologic condition.

Embodiment 34: The method of embodiment 33, wherein the benign gynecologic conditions is endometriosis, adenomyosis, fibroids, or a combination thereof.

Embodiment 35: The method of embodiment 33 or embodiment 34, wherein the biological sample comprises a cervicovaginal lavage (CVL) sample, a urine sample, a vaginal swab or vaginal fluid, or cervicovaginal secretion; wherein the cervicovaginal secretion is collected via a self collected lavage or a menstrual cup.

Embodiment 36: A method of treating adenomyosis in a patient in need thereof, the method comprising: a) diagnosing adenomyosis in the patient by: i) obtaining a biological sample from the patient; ii) measuring the levels of five or more biomarkers in the sample obtained in (i); and iii) diagnosing the patient with adenomyosis if the patient has levels of at least five or more biomarkers altered from a predetermined threshold; and b) administering a therapeutic amount of a treatment to the patient if the patient is diagnosed with adenomyosis.

Embodiment 37: A method of treating adenomyosis in a patient in need thereof, the method comprising: a) diagnosing adenomyosis in the patient by: i) obtaining a biological sample from the patient; ii) measuring the levels of five or more biomarkers in the sample obtained in (i); and iii) diagnosing the patient with adenomyosis if the patient has levels of at least five or more biomarkers altered from a predetermined threshold; and b) administering a therapeutic amount of a treatment to the patient if the patient is diagnosed with adenomyosis.

Embodiment 38: A method of treating adenomyosis in a patient in need thereof, the method comprising: a) diagnosing adenomyosis in the patient by: i) obtaining a cervicovaginal lavage (CVL) sample from the patient; ii) measuring the levels of five or more biomarkers in the sample obtained in (i); and iii) diagnosing the patient with adenomyosis if the patient has levels of at least five or more biomarkers altered from a predetermined threshold; and b) administering a therapeutic amount of a treatment to the patient if the patient is diagnosed with adenomyosis

Embodiment 39: The method of any one of embodiments 33-38, wherein the method comprises measuring the levels of ten or more biomarkers in the sample obtained in (i).

Embodiment 40: The method of any one of embodiments 33-38, wherein the method comprises measuring the levels of fifteen or more biomarkers in the sample obtained in (i).

Embodiment 41: The method of any one of embodiments 33-38, wherein the method comprises measuring the levels of twenty or more biomarkers in the sample obtained in (i).

Embodiment 42: The method of any one of embodiments 33-41, wherein the patient is diagnosed with adenomyosis if the levels of at least ten or more biomarkers are altered from a predetermined threshold.

Embodiment 43: The method of any one of embodiments 33-41, wherein the patient is diagnosed with adenomyosis if the levels of at least fifteen or more biomarkers are altered from a predetermined threshold.

Embodiment 44: The method of any one of embodiments 33-41, wherein the patient is diagnosed with adenomyosis if the levels of at least twenty or more biomarkers are altered from a predetermined threshold.

Embodiment 45: The method of any one of embodiments 33-44, wherein the five or more biomarkers comprise protein biomarkers, metabolite biomarkers, or a combination thereof.

Embodiment 46: The method of any one of embodiments 33-45, wherein the biomarkers comprise N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, phenethylamine, or a combination thereof.

Embodiment 47: The method of any one of embodiments 33-46, wherein the treatment comprises a nonsurgical treatment or a surgical treatment.

Embodiment 48: The method of embodiment 47, wherein the nonsurgical treatment comprises contraceptives, wherein the contraceptives comprise hormonal contraceptives.

Embodiment 49: The method of embodiment 47, wherein the surgical treatment comprises a hysterectomy.

Embodiment 50: A method of distinguishing between adenomyosis and endometriosis in a subject, the method comprising: a) obtaining a biological sample from the subject; and b) measuring the levels of five or more biomarkers in the sample obtained in (a); wherein the subject is determined to have adenomyosis if the levels of at least five or more biomarkers are altered from a predetermined threshold.

Embodiment 51: The method of embodiment 50, wherein the method comprises measuring the levels of ten or more biomarkers in the sample obtained in (a).

Embodiment 52: The method of embodiment 50, wherein the method comprises measuring the levels of fifteen or more biomarkers in the sample obtained in (a).

Embodiment 53: The method of embodiment 50, wherein the method comprises measuring the levels of twenty or more biomarkers in the sample obtained in (a).

Embodiment 54: The method of any one of embodiments 50-53, wherein the patient is diagnosed with adenomyosis if the levels of at least ten or more biomarkers are altered from a predetermined threshold.

Embodiment 55: The method of any one of embodiments 50-53, wherein the patient is diagnosed with adenomyosis if the levels of at least fifteen or more biomarkers are altered from a predetermined threshold.

Embodiment 56: The method of any one of embodiments 50-53, wherein the patient is diagnosed with adenomyosis if the levels of at least twenty or more biomarkers are altered from a predetermined threshold.

Embodiment 57: The method of any one of embodiments 50-56, wherein the biomarkers comprise protein biomarkers, metabolite biomarkers, or a combination thereof.

Embodiment 58: The method of any one of embodiments 50-57, wherein the five or more biomarkers comprise N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, phenethylamine, or a combination thereof.

Embodiment 59: The method of any of embodiments 50-58, wherein the biological sample comprises a cervicovaginal lavage (CVL) sample, a urine sample, a vaginal swab or vaginal fluid, or a cervicovaginal secretion; wherein the cervicovaginal secretion is collected via a self collected lavage or a menstrual cup.

Embodiment 60: An in vitro method of diagnosing a benign gynecologic condition in a subject in need thereof, the method comprising; a) producing a profile from a biological sample obtained from the subject by detecting at least five or more biomarkers; and b) analyzing the biological sample profile produced; wherein the subject is diagnosed with the benign gynecologic condition if the levels of at least five biomarkers are altered compared to a healthy control profile.

Embodiment 61: The method of embodiment 60, wherein the benign gynecologic conditions is endometriosis, adenomyosis, fibroids, or a combination thereof.

Embodiment 62: The method of embodiment 60 or embodiment 61, wherein the biological sample comprises a cervicovaginal lavage (CVL) sample, a urine sample, a vaginal swab or vaginal fluid, or cervicovaginal secretion; wherein the cervicovaginal secretion is collected via a self collected lavage or a menstrual cup.

Embodiment 63: An in vitro method of diagnosing adenomyosis in a subject in need thereof, the method comprising; a) producing a profile from a biological sample obtained from the subject by detecting at least five or more biomarkers; and b) analyzing the biological sample profile produced; wherein the subject is diagnosed with adenomyosis if the levels of at least five biomarkers are altered compared to a healthy control profile.

Embodiment 64: The method of embodiment 63, wherein the biological sample comprises a cervicovaginal lavage (CVL) sample, a urine sample, a vaginal swab or vaginal fluid, or cervicovaginal secretion; wherein the cervicovaginal secretion is collected via a self collected lavage or a menstrual cup.

Embodiment 65: An in vitro method of diagnosing adenomyosis in a subject in need thereof, the method comprising; a) producing a profile from a cervicovaginal lavage (CVL) sample obtained from the subject by detecting at least five or more biomarkers; and b) analyzing the CVL sample profile produced; wherein the subject is diagnosed with adenomyosis if the levels of at least five biomarkers are altered compared to a healthy control profile.

Embodiment 66: The method any one of embodiments 60-65, wherein producing a profile comprises detecting at least ten or more biomarkers.

Embodiment 67: The method any one of embodiments 60-65, wherein producing a profile comprises detecting at least fifteen or more biomarkers.

Embodiment 68: The method any one of embodiments 60-65, wherein producing a profile comprises detecting at least twenty or more biomarkers.

Embodiment 69: The method of any one of embodiments 60-68, wherein the patient is diagnosed with adenomyosis if the levels of at least ten or more biomarkers are altered from a predetermined threshold.

Embodiment 70: The method of any one of embodiments 60-68, wherein the patient is diagnosed with adenomyosis if the levels of at least fifteen or more biomarkers are altered from a predetermined threshold.

Embodiment 71: The method of any one of embodiments 60-68, wherein the patient is diagnosed with adenomyosis if the levels of at least twenty or more biomarkers are altered from a predetermined threshold

Embodiment 72: The method of any one of embodiments 60-71, wherein the biomarkers comprise protein biomarkers, metabolite biomarkers, or a combination thereof.

Embodiment 73: The method of any one of embodiments 60-72, wherein the five or more biomarkers comprise N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, phenethylamine, or a combination thereof.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A non-invasive method of diagnosing a benign gynecologic condition in a patient, the method comprising:
a) determining the patient's levels of five or more biomarkers comprising protein biomarkers, metabolites biomarkers, or a combination thereof by:
i) obtaining a biological sample from the patient; and
ii) measuring the levels of five or more biomarkers in the sample obtained in (i); and
b) diagnosing the patient with the benign gynecologic conditions if the patient has levels of at least five or more biomarkers are altered from a predetermined threshold.

2. The method of claim 1, wherein the benign gynecologic conditions is endometriosis, adenomyosis, fibroids, or a combination thereof.

3. The method of claim 1, wherein the biological sample comprises a cervicovaginal lavage (CVL) sample, a urine sample, a vaginal swab, or a cervicovaginal secretion; wherein the cervicovaginal secretion is collected via a self collected lavage or a menstrual cup.

4. The method of claim 1 wherein the method comprises measuring the levels of ten or more biomarkers in the sample obtained in (i).

5. The method of claim 4, wherein the patient is diagnosed with adenomyosis if the levels of at least ten or more biomarkers are altered from a predetermined threshold.

6. The method of claim 1, wherein the five or more biomarkers are selected from a group comprising N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, or phenethylamine.

7. The method of claim 1, wherein the five or more biomarkers are upregulated in the benign gynecologic condition from a predetermined threshold.

8. A method of treating a benign gynecologic condition in a patient in need thereof, the method comprising:
a) diagnosing the benign gynecologic condition in the patient by:
i) obtaining a biological sample from the patient;
ii) measuring the levels of five or more biomarkers comprising protein biomarkers, metabolites biomarkers, or a combination thereof in the sample obtained in (i); and
iii) diagnosing the patient with the benign gynecologic condition if the patient has levels of at least five or more biomarkers altered from a predetermined threshold; and
b) administering a therapeutic amount of a treatment to the patient if the patient is diagnosed with a benign gynecologic condition.

9. The method of claim 8, wherein the benign gynecologic conditions is endometriosis, adenomyosis, fibroids, or a combination thereof.

10. The method of claim 8, wherein the biological sample comprises a cervicovaginal lavage (CVL) sample, a urine sample, a vaginal swab or vaginal fluid, or cervicovaginal secretion;
wherein the cervicovaginal secretion is collected via a self collected lavage or a menstrual cup.

11. The method of claim 8, wherein the method comprises measuring the levels of ten or more biomarkers in the sample obtained in (i).

12. The method of any one of claims 11, wherein the patient is diagnosed with adenomyosis if the levels of at least ten or more biomarkers are altered from a predetermined threshold.

13. The method of claim 8, wherein the five or more biomarkers are selected from a group comprising N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, or phenethylamine.

14. The method of claim 8, wherein the treatment comprises a nonsurgical treatment or a surgical treatment.

15. The method of claim 14, wherein the nonsurgical treatment comprises contraceptives, wherein the contraceptives comprise hormonal contraceptives.

16. The method of claim 14, wherein the surgical treatment comprises a hysterectomy.

17. A method of distinguishing between adenomyosis and endometriosis in a subject, the method comprising:
a) obtaining a biological sample from the subject; and
b) measuring the levels of five or more biomarkers comprising protein biomarkers, metabolites biomarkers, or a combination thereof in the sample obtained in (a);
wherein the subject is determined to have adenomyosis if the levels of at least five or more biomarkers are altered from a predetermined threshold.

18. The method of claim 17, wherein the method comprises measuring the levels of ten or more biomarkers in the sample obtained in (a), wherein the patient is diagnosed with adenomyosis if the levels of at least ten or more biomarkers are altered from a predetermined threshold.

19. The method of claim 17, wherein the five or more biomarkers are selected from a group comprising N-carbamoylsarcosine, Fibrinopeptide A, N-methylhydroxyproline, N-methylproline, X-21803, carbohydrate antigen 19-9 (CA19-9), carcinoembryonic antigen (CEA), growth regulated oncogene (GRO), interferon γ-induced protein 10 kDa (IP-10), Interleukin (IL)-9, IL-13, IL-36γ, IL-17A, SHBG, tumor necrosis factor-beta (TNFβ), N6-acetyllysine, N-formylmethionine, argininate, pipecolate, 2-hydroxyadipic, N6,N6,N6-trimethyllysine, N-alpha-acetylornithine, 3-formylindole, oxalate, X-11615, X-21803, 3′-dephospho-acetyl-CoA, or phenethylamine.

20. The method of claim 16, wherein the biological sample comprises a cervicovaginal lavage (CVL) sample, a urine sample, a vaginal swab or vaginal fluid, or a cervicovaginal secretion; wherein the cervicovaginal secretion is collected via a self collected lavage or a menstrual cup.
